(19) United States
(12) Patent Application Publication
(10) Pub. No.: US 2024/0105284 A1
(43) Pub. Date: Mar. 28, 2024

(54) USER INTERFACE AND BACKEND SYSTEM FOR PATHOGEN ANALYSIS

(71) Applicant: CZ Biohub SF, LLC, San Francisco, CA (US)

(72) Inventors: David Dynerman, San Francisco, CA (US); Lucy Li, San Francisco, CA (US)

(21) Appl. No.: 17/768,780

(22) PCT Filed: Nov. 5, 2020
(86) PCT No.: PCT/US2020/059190

§ 371 Date: Apr. 13, 2022

Related U.S. Application Data
(60) Provisional application No. 62/931,778, filed on Nov. 6, 2019.

(51) Int. Cl. G16B 50/30; C12Q 1/6888; G16B 20/00; G16B 30/00

(52) U.S. Cl. CPC G16B 50/30; C12Q 1/6888; G16B 20/00; G16B 30/00

(57) ABSTRACT

Techniques for a user interface for pathogen analysis are provided. The user interface may include an interactive dendrogram that identifies a plurality of biological samples and their corresponding sequences. The biological samples of the interactive dendrogram may be arranged based on a degree of similarity between nucleotide sequences of the biological samples. In response to a selection of biological sample, the interactive dendrogram may identify a cluster of biological samples. Each biological sample of the cluster can be identified based on a determination that a number of variations between the sequences of the biological sample and the selected biological samples are under a predefined threshold. The user interface may also include a similarity matrix that identifies a number of variations between sequences of two biological samples selected from the interactive dendrogram.

### CROSS-REFERENCES TO RELATED APPLICATIONS

The present application claims priority from and is a PCT application of U.S. Provisional Application No. 62/931,778, entitled “User Interface And Backend System For Pathogen Analysis” filed Nov. 6, 2019, the entire contents of which is herein incorporated by reference in its entirety for all purposes.

### FIELD

Methods and systems disclosed herein relate generally to a user interface system for processing and visualization of medical and genomic information relating to pathogens.

### BACKGROUND

Recent years have seen the development of tracking pathogens by sequence information in order to identify infectious disease outbreaks (Hadfield et al. 2018; CGPS 2018). To perform the epidemiological analysis of the pathogen sequences, health departments and hospitals in the United States of America generally use rudimentary software tools such as spreadsheets. Tracking millions of nucleotide sequences using such rudimentary software tools is cumbersome and error-prone. In addition, extracting actionable information from medical data (e.g., date, geographical location) associated with the nucleotide sequences can be challenging and difficult. In several instances, a user may need to review several types of medical data in various ways to identify infectious disease outbreaks that warrants resources to be expended.

### BRIEF SUMMARY

In some embodiments, computer-implemented methods are provided as described herein. For example, techniques for a user interface for pathogen analysis are provided. The user interface may include an interactive dendrogram that identifies a plurality of biological samples and their corresponding sequences. The biological samples of the interactive dendrogram may be arranged based on a degree of similarity between nucleotide sequences of the biological samples. In response to a selection of biological samples, the interactive dendrogram may identify a cluster of biological samples. Each biological sample of the cluster can be identified based on a determination that a number of variations between the sequences of the biological sample and the selected biological samples are under a predefined threshold. The user interface may also include a similarity matrix that identifies a number of variations between sequences of two biological samples selected from the interactive dendrogram.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

A better understanding of the nature and advantages of embodiments of the present disclosure may be gained with reference to the following detailed description and the accompanying drawings.

### BRIEF DESCRIPTION OF THE DRAWINGS

The present disclosure is described in conjunction with the appended figures:

FIG. 1 illustrates an example computing environment for detecting and tracking infectious disease outbreaks in accordance with some embodiments.

FIG. 2 illustrates a sequence diagram for uploading and processing biological-sample data in accordance with some embodiments.

FIG. 3 illustrates an example screenshot of an input interface for uploading files that identify biological samples collected from several facilities and regions.

FIG. 4 illustrates a process in which an input interface can be used to upload biological-sample files in accordance with some embodiments.

FIGS. 5A-C illustrate a first set of example screenshots of an interactive dendrogram of a user interface in accordance with some embodiments.

FIGS. 6A-C illustrate a second set of example screenshots of an interactive dendrogram that provides a more detailed view of a heatmap in accordance with some embodiments.

FIGS. 7A-B illustrate a third set of example screenshots of an interactive dendrogram that identifies clusters of biological samples in accordance with some embodiments

FIG. 8 illustrates a process in which a cluster of biological samples is identified from an interactive dendrogram in accordance with some embodiments.

FIGS. 9A-B illustrate a set of example screenshots of an interactive dendrogram and a similarity matrix in accordance with some embodiments.

FIG. 10 illustrates an example database for generating a similarity matrix in accordance with some embodiments.

FIG. 11 illustrates a process for configuring a similarity matrix in accordance with some embodiments.

FIG. 12 illustrates a process for restricting access to biological-sample data in accordance with some embodiments.

FIG. 13 shows a block diagram of an example computer system usable with systems and methods according to embodiments of the present invention.

### DETAILED DESCRIPTION

Techniques relate to systems and methods for processing and visualizing medical and genomic data corresponding to pathogens (e.g., bacteria, viruses, fungi). More specifically, a user interface is presented that can be used to manage files corresponding to biological samples and their respective nucleotide sequences. A sequence analyzer operating on a backend can access and process the files to enable clustering of biological samples that have similar nucleotide sequences. The user interface can also allow a user to interact with the processed biological sample and nucleotide sequences using various types of user-interface components. For example, a user-interface component may include an interactive dendrogram that can be used to indicate clusters of biological samples that have similar nucleotide sequences. Another user-interface component may include a similarity matrix that can be used to indicate a degree of similarity between nucleotide sequences corresponding to two or more biological samples that were selected via the user interface.

Accordingly, the user interface can generate, based on the user interactions, types of information that each indicate an extent of similarity between given biological samples. The generated information can then be used to enable detection of pathogens at a particular geographic region. The generated information can also be used to identify possible infectious disease outbreaks caused by such pathogens at the particular geographic region (Harris et al. 2010; Gardy & Loman 2018). Moreover, the medical and genomic data of pathogens collected from different regions and facilities can be stored in a database server. Based on a user's affiliation to a particular region and/or facility, certain parts of the medical and genomic data can be anonymized during the user session to protect unauthorized exposure of sensitive medical information.

### I. EXAMPLE COMPUTING ENVIRONMENT FOR PATHOGEN ANALYSIS

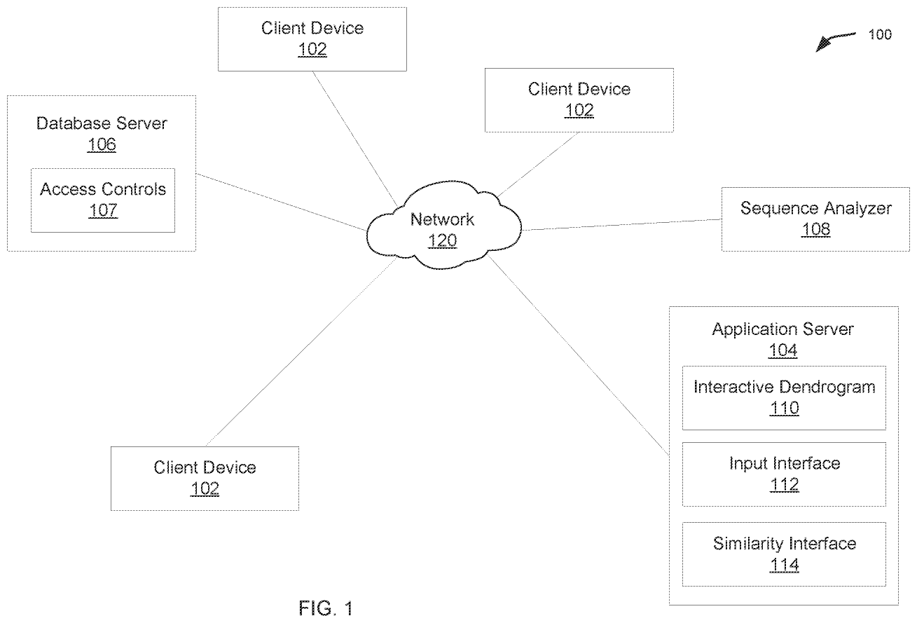

FIG. 1 illustrates an example computing environment 100 for detecting and tracking infectious disease outbreaks in accordance with some embodiments. The computing environment 100 may include various devices, systems, and/or components, such as a client device 102, an application server 104, a database server 106, and a sequence analyzer 108. In some instances, the application server 104, the database server 106, and the sequence analyzer 108 may collectively correspond to a single backend system that communicates with the client device 102. Although a single client device 102 is shown to interact with the systems in the computing environment 100, multiple client devices can access and interact with one or more of the application server 104, the database server 106, and the sequence analyzer 108.

The devices, systems, and/or components of the computing environment 100 can transmit network packets through a network 120 for network communication, data transfer, and the like. Further, the devices, systems, and/or components of the computing environment 100 can process data uploaded from a user, which can be displayed on a user interface of the client device 102. The user interface can include various user-interface elements that identify biological samples collected from various facilities and regions. In some instances, the user interface also includes user-interface elements that correspond to nucleotide sequences of the identified biological samples. Using input/output devices associated with the client device 102, a user may interact with one or more of the user-interface elements to identify genetic similarity between the biological samples. By facilitating analysis of the biological samples, infectious disease outbreaks can be detected, pathogens causing the infection disease outbreaks can be identified, and/or any infectious disease outbreaks potentially spreading towards different regions can be tracked.

A. Application Server

The application server 104 may include programmable components that can interact with an input interface 112 for uploading files that include biological samples collected from several facilities and regions. The application server 104 may receive a file that identifies a plurality of biological samples. As a result of the file being successfully uploaded, the application server 104 may receive a set of nucleotide-sequence files. The application server 104 can associate each biological sample identified in the file with a corresponding nucleotide-sequence file from the set. The associated biological samples can be stored in the database server 106. The stored data in the database server 106 can be accessed by the sequence analyzer 108, which can identify a gene corresponding to each sequence of the biological sample (for example). In some instances, the sequence analyzer 108 generates, for each biological sample, a set of results. Each result of the set may indicate a number of the SNPs between the biological samples and another biological sample that is accessed from the database server 106. The set of results for each biological sample can be stored back in the database server 106. The application server 104 can output the processed data stored in the database server 106 for displaying the biological samples and corresponding nucleotide sequences in the user interface of the client device 102 (for example). In some instances, the application server 104 may transmit the processed data through a web server (not shown), which may display the biological samples and corresponding nucleotide sequences on a web browser of the client device 102 (for example).

The application server 104 includes programmable components that enable the user interface to provide various interactive user-interface elements for investigating infectious disease outbreaks. The application server 104 may include an interactive dendrogram 110 for the user interface. The interactive dendrogram 110 may cluster biological samples that have similar nucleotide sequences, at which the biological samples with the least number of SNPs can be positioned next to each other (for example). The application server 104 may also include a similarity interface 114, which can generate a similarity matrix that indicates a number of SNPs between biological samples selected through the user interface. In some instances, the biological samples are selected by interacting with one or more sample identifiers displayed on the interactive dendrogram 110. The application server 104 may also restrict certain types of data to be displayed on the user interface. For example, facility names associated with a set of biological samples can be replaced with generic identifiers when the set of biological samples are displayed on the interactive dendrogram 110. Alternatively or additionally, the application server 104 may avail a user to download any or all of the stored records in the database server 106.

1. Input Interface

The application server 104 may include programmable components to generate the input interface 112 that allows a user to upload a file that includes metadata (e.g., identifiers and locations of a sample) for biological samples collected at a given facility of a geographical region. In some instances, the file is uploaded by drag-and-dropping the file on a first area of the input interface 112. The input interface 112 can also include a second area at which a set of nucleotide-sequence files can be uploaded. The uploaded file storing the metadata for the biological samples can be further processed, as well as sequence data that is linked to the metadata. In particular, each biological sample of the file can be associated with an uploaded nucleotide-sequence file that includes a nucleotide sequence corresponding to the biological sample. The biological samples and their respective nucleotide sequences can then be stored as records of a database. Additionally or alternatively, the file can be previewed through the input interface 112 prior to upload, so as to allow the user to identify any sensitive information that may have accidentally been stored in the file.

2. Interactive Dendrogram

The application server 104 may include programmable components usable to generate the interactive dendrogram 110 which can be displayed on a user interface and can be used to analyze sequences corresponding to biological samples. The interactive dendrogram 110 may include two interactive parts. A first part of the interactive dendrogram 110 may display a first plurality of biological-sample identifiers as user-interface elements. Each sample identifier corresponds to a biological sample associated with a database record. A sample identifier may include an alphanumerical string that indicates a name of a nucleotide-sequence file, a location from which the biological sample was collected (e.g., facility, hospital), and a date at which the biological sample was collected.

A second part of the interactive dendrogram 110 may display a table for whether a sample sequence includes a particular gene, where the table includes a second plurality of user-interface elements that correspond to each sample identifier. Accordingly, each of the second plurality may identify a name of a gene and indicate whether the corresponding biological sample includes the identified gene. The presence of a gene can be determined by the sequence analyzer 108 that can compare the nucleotide sequence of the biological sample to a nucleotide sequence corresponding to the gene that is indicated by the user-interface element. In addition, the interactive dendrogram 110 may present the sample identifiers in a manner that biological samples that have similar nucleotide sequences can be clustered together.

The interactive dendrogram 110 may receive a user selection of a biological sample. In response to the user selection, the interactive dendrogram 110 may highlight a cluster of biological samples that are considered to have similar nucleotide sequences. To determine whether a given biological sample has a similar sequence to that of the selected biological sample, a number of SNPs can be counted between the two biological samples. If the number of SNPs is under a predetermined threshold, the given biological sample may be added into the cluster of biological samples. On the other hand, if the number of SNPs exceeds the predetermined threshold, the given biological sample may be excluded or otherwise removed from the cluster of biological samples. Biological samples can be clustered using various sequence-alignment techniques, including sequence alignment techniques described in Li et al. 2009 and pairwise alignment techniques described in Li 2018.

The predetermined threshold can indicate an upper limit of SNPs when determining similarity of nucleotide sequences between the two biological samples. The predetermined threshold can be modified based on adjusting an interactive user-interface element (e.g., a range slider, a text box) of the interactive dendrogram 110. Modifying the predetermined threshold may result in an identification of a different cluster of biological samples. For example, modifying the predetermined threshold to a greater value can result in a larger cluster of biological samples relative to the cluster identified at initial threshold. In another example, modifying the predetermined threshold to a lesser value can result in a smaller cluster of biological samples relative to the cluster identified at initial threshold.

3. Similarity Interface

In addition to the interactive dendrogram 110, the application server 104 may further include the similarity interface 114. The similarity interface 114 can be used to generate a similarity matrix that can provide metrics corresponding to a degree of similarity between sequences corresponding to biological samples that are presented in the interactive dendrogram 110. The metric can indicate a number of SNPs between biological samples selected by the user. For each biological sample selected through the interactive dendrogram 110, matrix elements can be added such that dimensions corresponding the similarity matrix 114 can increase by 1. For example, when two biological samples are selected from the interactive dendrogram 110, the similarity matrix 114 with 2×2 dimensions may be generated.

Additional biological samples can be selected after the initial similarity matrix 114 is generated. When an additional biological sample is selected, similarity interface 114 may add matrix elements can be added to the similarity matrix such that the dimensions of the similarity matrix 114 can increase in proportion to the number of the additional biological samples. For example, a similarity matrix 114 with 3×3 dimensions can increase to 5×5 dimensions, in response to two additional biological samples being selected from the interactive dendrogram 110. Conversely, biological samples in the similarity matrix can be unselected, at which matrix elements can be removed from the similarity matrix such that the dimensions of the similarity matrix can decrease in proportion to the number of biological samples that were unselected. In some instances, the similarity interface 114 generates, for the similarity matrix, an average value of SNPs among the biological samples of the similarity matrix (for example) and a number of SNPs that corresponds to an unselected biological sample that has the most similar sequence to those of the biological samples of the similarity matrix.

B. Database Server

The database server 106 may receive biological-sample files and nucleotide-sequence files from the application server 104. The database server 106 may store the received files in one or more databases, which can later accessed by other systems of the computing environment 100. For example, the application server 104 may access the data stored in the database server 106 to generate the interactive dendrogram 110 for investigating infectious disease outbreaks. In another example, the sequence analyzer 108 may access the stored data to generate additional data corresponding to the biological samples, including the number of SNPs between two biological samples. The database server 106 may be configured to interact with data stored in the database based on using various types of database queries (e.g., insert, update, select, delete). In some instances, the database server 106 provides an application programming interface (API) to allow other systems to store and access the data based on a set of API parameters. The database server 106 may also be configured to store data in a relational (e.g., SQL) and/or a non-relational (e.g., NoSQL) database.

As the biological-sample files are transmitted by the application server 104, the database server 106 may generate a new database record for each biological sample stored in the file. The new database records that correspond to the biological-sample files can be stored in a global database table. The global database table may include biological samples across all regions and groups. For each record of the global database table, the database server 106 may also store a nucleotide-sequence file that corresponds to the biological sample of the record. The database records including the biological samples and respective nucleotide sequences can be accessed by the sequence analyzer 108, which may generate additional data that may provide another context for analyzing pathogens detected in different regions. For example, the sequence analyzer 108 may identify genetic-variation metrics (e.g., a number of SNPs) between a biological sample and each of the other biological samples stored in the global database table. The identification operations may repeat through each biological sample of the global database table, in which genetic-variation metrics can be identified between the biological sample and each of all other biological samples.

The sequence analyzer 108 may store the identified genetic-variation metrics in another database table of the database server 106, which can be used by the interactive dendrogram 100 and/or the similarity interface 114 of the user interface. To reduce storage redundancy, the data generated by the sequence analyzer 108 may be stored once rather than storing the same data repeatedly. In addition, the database server 106 may transmit stored database records corresponding to a subset of the biological samples stored in the global database table, at which a local database table can be generated from the transmitted records. The user interface of a device (e.g., the client device 102) can use the local database table to retrieve various data (e.g., genetic-variation metrics between two biological samples) and generate user-interface elements based on the retrieved data. Generating a local database table may reduce network latency and decrease processing time associated with generating a report for the user.

1. Access Controls

The database server 106 may receive biological samples and nucleotide sequences uploaded by multiple users from respective regions and groups. As a result, the database server 106 can be accessed to provide information across regions to facilitate tracking of infectious disease outbreaks. To prevent sensitive information (e.g., name of the facility, consortium) from being shared by unauthorized users, the database server may apply access controls 107, which access to data corresponding to one or more categories can be restricted. The access controls 107 may indicate an extent of the restriction, which can be determined (for example) based on the database server 106 comparing a user-affiliated group to a group associated with an entity that uploaded the data, a user-affiliated consortium to a consortium associated with the entity that uploaded the data, a user-affiliated region to a region associated with the entity that uploaded the data, and so on.

For example, if an accessing user is affiliated to the group that matches the group of an entity that uploaded the biological samples, the database server may provide the biological samples and corresponding nucleotide sequences without any restriction. In another example, if the accessing user is affiliated with a different group but the same consortium as those of the entity that uploaded the biological samples, the database server may restrict information that could reveal the group name of the entity. Based on the extent of the restriction, the database server can redact the information from the user interface (for example) or replace the information with other generic information (for example).

C. Sequence Analyzer

The sequence analyzer 108 can include several components to detect and analyze sequences corresponding to the biological samples stored in the database server 106. The sequence analyzer 108 can access each added record that includes a biological sample and its corresponding nucleotide sequence. The nucleotide sequence corresponding to the biological sample can be submitted as a query, such that the sequence analyzer 108 can identify a corresponding gene for each sub-sequence of the nucleotide sequence. In some instances, the sequence is converted to a file format (e.g., FASTA format) that can be processed by the sequence analyzer 108. In some instances, the sequence analyzer 108 identifies a set of genes based on the sub-sequences of the nucleotide sequence corresponding to the biological sample. The set of genes can be used to further identify a pathogen and/or a strain that corresponds to such pathogen. For a gene of the set of genes, the sequence analyzer 108 may additionally indicate whether the gene is antibiotic resistant. Antibiotic-resistant genes can be identified using various techniques, including molecular typing techniques described in Inouye et al. 2014. Based on the gene and strain identification of the biological samples, a user can determine whether biological samples collected from different regions correspond to the same strain which may indicate transmission of infectious diseases. The database can then be updated by adding the identified genes to the database record of the database server 106.

In addition to identifying genes that correspond to the nucleotide sequence, the sequence analyzer 108 may also generate a set of results for each biological sample, in which each result of the set corresponds to a genetic-variation metric corresponding to a number of sequence variations (e.g., single nucleotide polymorphisms (SNPs)) between the biological sample and another biological sample that is stored in the database. The set of SNPs for each biological sample can be generated such that biological samples that have similar nucleotide sequences can be clustered together.

The genetic-variation metrics for the biological samples can also be used for a similarity matrix that corresponds to the biological samples. In addition, hierarchical clustering can be performed on the genetic-variation metrics to generate an interactive dendrogram 110. As described herein, the interactive dendrogram 110 may include clusters of biological samples that are formed based on similarities of nucleotide sequences. In this manner, transmissions or outbreaks of infectious diseases can be detected and tracked based on the output generated by the sequence analyzer 108. For example, if a biological sample from a region and another biological sample from a proximate region correspond to identical nucleotide sequences, there may be a strong indication that a transmission of the disease may have occurred. Conversely, two biological samples corresponding to a very high number of SNPs may indicate that there is a low likelihood of disease transmission. In some instances, the genetic-variation metrics output by the sequence analyzer 108 are used to determine whether two samples belong to same or different strains corresponding to a pathogen species (for example).

D. Sequence Diagram

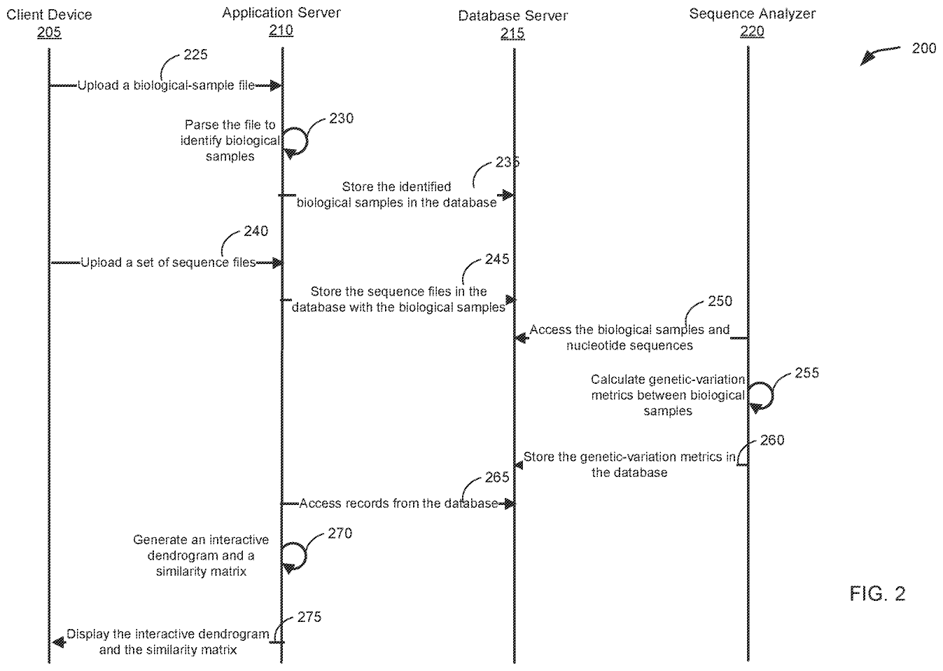

FIG. 2 illustrates a sequence diagram 200 for uploading and processing biological-sample data in accordance with some embodiments. A client device 205 may initiate an upload process by uploading a file that includes metadata (e.g., identifiers and locations of a sample) for biological samples collected at a given facility of a geographical region (step 225). An application server 210 (e.g., the application server 104 of FIG. 1) may parse the metadata of the biological-sample file to identify a plurality of biological samples (step 230). The application server 210 may store the plurality of biological samples in the database server 215, in which a database record can be generated for each biological sample of the plurality (step 235).

The client device 205 can then upload a set of nucleotide-sequence files to the application server 210 (step 240). Each nucleotide-sequence file of the set of nucleotide-sequence files may include nucleotide sequences corresponding to a biological sample identified by the biological-sample file. The application server 210 may store the uploaded set of nucleotide-sequence files in the database server 215 (step 245). To store the nucleotide-sequence files, the database server 215 may associate a database record with an uploaded nucleotide-sequence file, in which the nucleotide-sequence file that includes a nucleotide sequence corresponding to the biological sample indicated by the database record.

The sequence analyzer 220 may access the database records that identify the biological-samples and corresponding nucleotide sequence (step 250). The sequence analyzer 220 may process the database records to calculate genetic-variation metrics between a biological sample and each of other biological samples stored in the database server 215 (step 255). In some instances, hierarchical clustering is performed on the genetic-variation metrics such that an interactive dendrogram can be generated for the client device 205. The sequence analyzer 220 may then store the genetic-variation metrics in the database of the database server 215 (step 260).

The application server 210 may access the stored records from the database server 215 (step 265). The stored records may include information identifying the biological samples and the corresponding nucleotide sequences, as well as genetic-variation metrics for the biological samples. Based on the retrieved information, the application server 210 may generate the interactive dendrogram and the similarity matrix (step 270). The interactive dendrogram may include clusters of biological samples that are formed based on similarities of their respective nucleotide sequences. The similarity matrix that can be used to indicate a degree of similarity between nucleotide sequences corresponding to two or more biological samples that were selected via the user interface. After the interactive dendrogram and the similarity matrix are generated, the application server 210 may cause the interactive dendrogram and the similarity matrix to be displayed on a user interface (e.g., a web browser) of the client device 205 (step 275).

### II. INPUT INTERFACE FOR PATHOGEN ANALYSIS

A. Overview

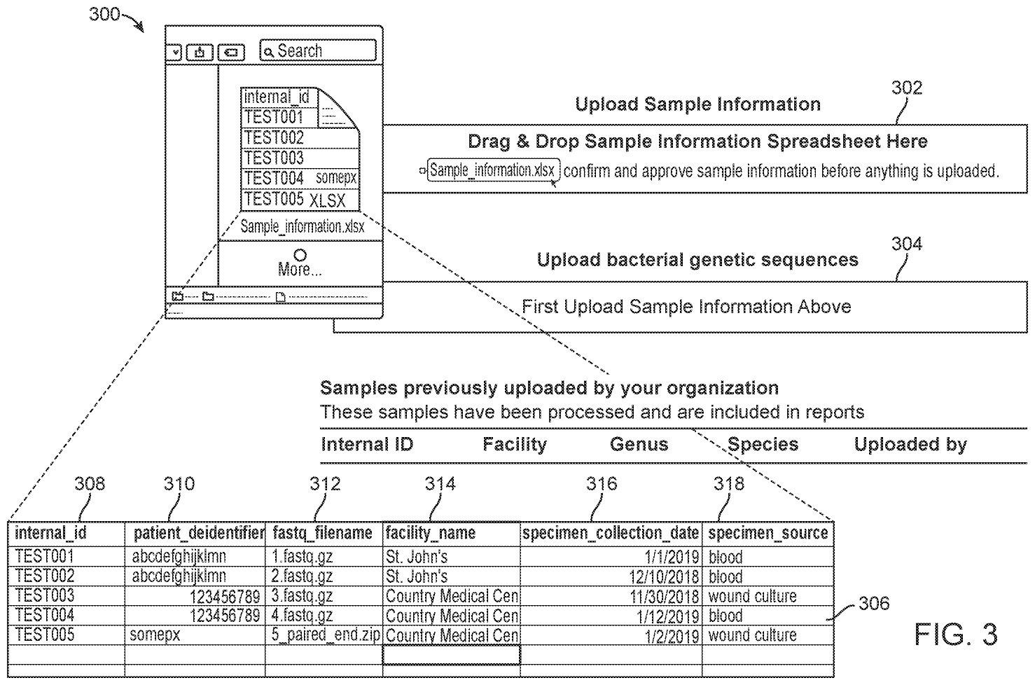

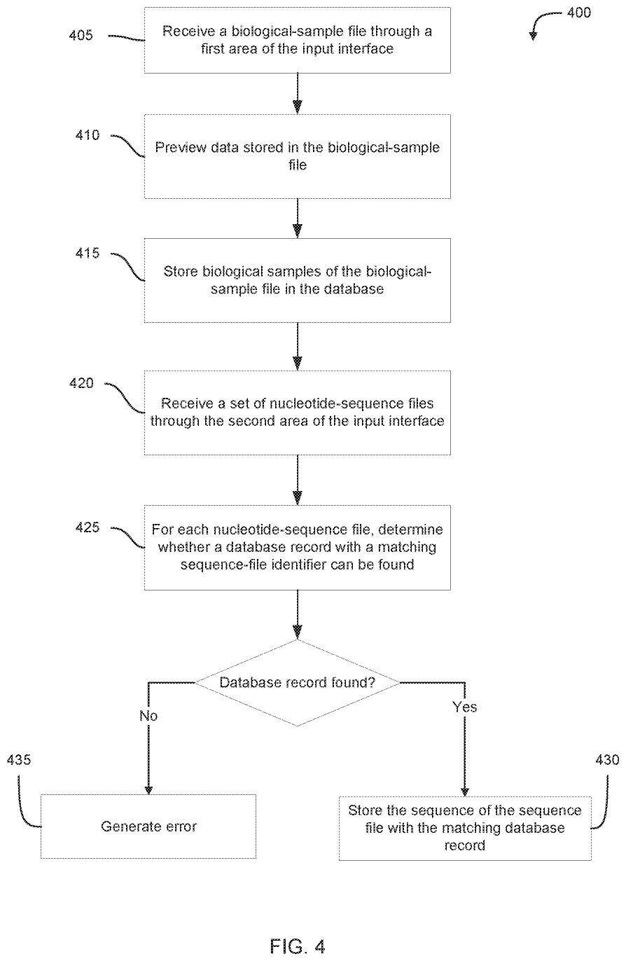

FIG. 3 illustrates an example screenshot of an input interface 300 for uploading files that identify biological samples collected from several facilities and geographical regions. The input interface 300 may include a first area 302 and a second area 304. A biological-sample file 306 that identifies a plurality of biological samples can be uploaded through the first area 302. The biological-sample file 306 may include columns of metadata, including a sequence-file name, a date on which the biological sample was collected, and a specimen source. Uploading of the biological-sample file 306 may be initiated as a result of drag-and-dropping the biological-sample file 306 onto the first area 302. A database record can be generated for each biological sample listed in the biological-sample file 306. The database records can be stored in a database server (e.g., the database server 106 of FIG. 1). As a result of the biological-sample file 306 being successfully uploaded, the a set of nucleotide-sequence files (not shown) can be uploaded through the second area 304 of the input interface 300. Each nucleotide-sequence file of the set can be stored as part of a database record of corresponding biological sample.

In some instances, the data stored in the biological-sample file 306 are displayed as a preview at a display region of the input interface 300. The preview of the biological-sample file 306 can be overlaid on the first area 302 of the input interface 300. In this manner, a user can visually inspect that the biological-sample file 306 is the correct file for upload. A user can also ensure that no private or sensitive data was inadvertently included in the biological-sample file 306. In response to the set of nucleotide-sequence files being uploaded through the second area 304, the display region of the input interface 300 can also indicate that the set of nucleotide-sequence files does include nucleotide-sequence files that match the information as listed in the biological-sample file 306.

B. Configuration of Input Interface

As described herein, the input interface 300 may include the first area 302 on which a user can upload the biological-sample file 306. A user-interface operation to upload the biological-sample file 306 may include a drag-and-drop operation, a dialog box (e.g., pop-up) operation to browse and locate the biological-sample file 306, and copy-paste operation of the biological-sample file 306 to the first area 302. The biological-sample file 306 may include information usable to identify a plurality of biological samples, and each biological sample can be associated with a set of columns.

Each column of the set of columns may describe a type of characteristics corresponding to the biological sample. For example, a biological sample in the biological-sample file 306 may include the following columns: (i) a first column 308 indicating an internal identifier corresponding to the biological sample; (ii) a second column 310 indicating an identifier corresponding to a subject from which the biological sample was collected; (iii) a third column 312 indicating a sequence-file identifier that identifies a nucleotide-sequence file; (iv) a fourth column 314 indicating a name of a facility that collected the biological sample; (v) a fifth column 316 indicating a date at which the biological sample was collected; and (vi) a sixth column 318 indicating a source from which the biological sample was collected (e.g., blood, wound culture). The nucleotide-sequence file indicated in a column of the biological-sample file may include a nucleotide sequence that correspond to the biological sample. Alternatively or additionally, the set of columns in the biological-sample file 306 may include a column indicating a species, a genus, and/or a class of a pathogen that corresponds to the biological sample.

In some instances, the input interface 300 allows a user to preview data stored in the biological-sample file 306 before it is uploaded and stored in the database server. The input interface 300 may process the biological-sample file 306 and display contents of the biological-sample file 306 on the input interface 300. In addition to the displayed contents, the input interface 300 may also provide a prompt to confirm the submission of the biological-sample file 306. The displayed contents can be visually inspected by the user to ensure that private, sensitive data will be removed and the correct file will be uploaded to the database server. Once confirmed, the biological-sample file 306 can be uploaded to the database server.

During upload of the biological-sample file 306 to the database server, a database record can be generated for each biological sample identified in the biological-sample file 306. The database record may include at least some columns that correspond to the set of columns. Each column header of the set of columns of the biological-sample file 306 may be mapped to a corresponding column of the database record, at which the data corresponding to the set of columns of the biological-sample file 306 can be processed based on the mapping and copied onto the corresponding columns of the database record. In some instances, the database records includes at least some columns that are empty and will be populated with data generated from another system, such as a sequence analyzer (e.g., the sequence analyzer 108 of FIG. 1). As such, the empty columns of a database record can serve as placeholders for future data. Each of the database records can also be marked with an indicator, which indicates that the database record needs to be associated with a nucleotide-sequence file corresponding to the biological sample. As a result of being populated with data from the biological-sample file 306, the database records can be stored in the database server.

The user interface 300 may upload the set of nucleotide-sequence files through the second area 304. Each nucleotide-sequence file of the set of nucleotide-sequence files may include a nucleotide sequence corresponding to a biological sample of the biological-sample file 306. The nucleotide-sequence file may store the nucleotide sequence in a specific file format so as to allow a sequence analyzer to process the nucleotide sequence for analysis. A nucleotide sequence may include a plurality of nucleotide sub-sequences. For a given nucleotide sub-sequence of the nucleotide sequence, the specific file format (e.g., a FASTQ file format) may include a label identifying the nucleotide sub-sequence, a set of nucleotide symbols that correspond to the nucleotide sub-sequence, and a set of quality symbols indicating accuracy of the set of nucleotide symbols.

Similar to the biological-sample file 306, the set of nucleotide-sequence files can be uploaded through different types of user-interface operations including the drag-and-drop operation on the second area 304, the dialog box (e.g., pop-up) operation, and/or the copy-paste operation to the second area 304. To upload the set of nucleotide-sequence files, the file name of the nucleotide-sequence files can be compared to sequence-file identifiers stored in the database records. For example, a file identifier of the nucleotide-sequence file can be constructed as a database query. The database query can be submitted to identify a database record that includes a column value corresponding to the file identifier. The query process can be iterated through all nucleotide-sequence files to ensure that database records can be populated with nucleotide-sequence data,

If a nucleotide-sequence file that matches a sequence-file identifier is found, the nucleotide-sequence file can be stored with a database record associated with the matching sequence-file identifier. Conversely, if a nucleotide-sequence file does not match any of the sequence-file identifiers of the database records, the input interface 300 may issue an error message indicating unsuccessful upload of one or more nucleotide-sequence files. In some instances, the input interface 300 issues another error message in response to determining that a nucleotide-sequence file has not been identified and uploaded for at least one database record. If the nucleotide-sequence files are successfully linked to each of the database records, the input interface 300 may indicate successful upload of the biological samples and the nucleotide sequences.

C. Method for Input Interface

FIG. 4 illustrates a process 400 in which an input interface can be used to upload biological-sample files in accordance with some embodiments. Process 400 may be performed by the input interface 112 of FIG. 1. A web interface may be provided to a client device for providing data related to biological samples.

At step 405, when the input interface (e.g., the input interface 300 of FIG. 3) may receive a biological-sample file through its first area. The biological-sample file (e.g., the biological file 306 of FIG. 3) may include a plurality of sample identifiers usable to identify biological samples. In some instances, the biological-sample file includes a set of columns that include metadata (e.g., identifiers and locations of a sample) for biological samples collected at a given facility of a geographical region. The metadata corresponding to a sample identifier may also include a sequence-file identifier, which is usable to identify a nucleotide-sequence file that corresponds to the biological sample indicated by the sample identifier. In some instances, the biological-sample file is received via a drag-and-drop action performed via the input interface.

At step 410, data stored in the biological-sample file can be displayed as a preview. Based on the preview, the biological-sample file can be authorized for upload into a database. In some instances, the user aborts the upload process based on preview of the data. The abort operation can be due to presence of sensitive information in the biological-sample file. Displaying the data stored in the biological-sample file may include displaying, via the input interface, the plurality of sample identifiers in the first area of the graphical user interface. In some instances, metadata corresponding to the biological samples is additionally displayed as part of the preview in the first area.

At step 415, if the upload of the biological-sample file is authorized based on the preview, the biological-sample file is stored in a database server. To store the biological-sample file, a database record can be generated for each biological sample identified by the biological-sample file. The database record may include at least some columns that correspond to the set of columns of the biological-sample file. Each column header of the set of columns of the biological-sample file may be mapped to a corresponding column of the database record, at which the data corresponding to the set of columns can be processed based on the mapping and copied onto the corresponding columns of the database record.

At step 420, the input interface may receive a set of nucleotide-sequence files through its second area. Each nucleotide-sequence file may include a nucleotide sequence that correspond to a particular strain of a pathogen. Similar to the biological-sample file, the set of nucleotide-sequence files may be received via a drag-and-drop action performed via the input interface. The nucleotide sequence stored in the nucleotide-sequence file may include a plurality of nucleotide sub-sequences, in which each sub-sequence may indicate a gene of a pathogen.

At step 425, for each nucleotide-sequence file of the uploaded nucleotide-sequence files, a file identifier of the nucleotide-sequence file can be used to determine whether a database record with a matching sequence-file identifier can be found. For example, a file identifier of the nucleotide-sequence file can be constructed as a database query. The database query can be submitted to identify a database record that includes a column value corresponding to the file identifier. The query process can be iterated through all nucleotide-sequence files to ensure that database records can be populated with nucleotide-sequence data.

At step 430, in response to the database record being found (“Yes” branch of step 425), the nucleotide sequences of the nucleotide-sequence file can be stored with the database record. The stored database record can be displayed by a user interface. In some instances, the stored database record is displayed on another portion of the web interface displaying the input interface. The stored database record can also be retrieved by a sequence analyzer (e.g., the sequence analyzer 108 of FIG. 1) to perform additional analysis on the nucleotide sequence corresponding to the stored database record.

At step 435, in response to the database record not being found (“No” branch of step 425), the input interface can generate an error message. In addition to generating the error message, the process 400 can be aborted altogether. In some instances, the input interface may present the error message and another message that instructs the user to upload a new set of nucleotide-sequence files. As a result of a submission of the new set of nucleotide-sequence files, the process 400 can be re-initiated from step 425.

Step 425 and steps 430 or 435 can be iterated through the remaining nucleotide-sequence files of the set of nucleotide-sequence files, until either an error is issued or nucleotide sequences corresponding to all nucleotide-sequence files are processed. The input interface may indicate that the upload has been successful. In the event that one or more errors are generated, the input interface may generate a log indicating the one or more errors then display the log to the user. Biological samples corresponding to the database records can be displayed to the user. In some instances, one or more database records can be highlighted to indicate that the biological samples corresponding to the highlighted database records relate to a suspected infectious disease outbreak.

### III. INTERACTIVE DENDROGRAM

A. Overview

An interactive dendrogram can show biological samples that are clustered based on their nucleotide sequences. To generate the interactive dendrogram, nucleotide sequences corresponding to biological samples uploaded from an input interface (e.g., the input interface of FIG. 3) can be processed using a hierarchical clustering algorithm. By processing the nucleotide sequences through hierarchical clustering, biological samples that have similar sequences can be clustered together. In some instances, a cluster of biological samples in the interactive dendrogram is visually indicated (e.g., “red”) as biological samples that contribute to a suspected infectious disease outbreak.

In addition, the interactive dendrogram may include user-interface elements that may be used to visually indicate biological samples having nucleotide sequences that are substantially similar to those of a given biological sample. For example, a row of the plurality of rows can be selected (e.g., click, hover) by a user of the user interface. The selection may highlight the row with a first color (e.g, red). As the row is selected, a cluster of biological samples can be highlighted in which each biological sample in the cluster has a nucleotide sequence that are the same or substantially similar (e.g., as defined by a threshold) to the nucleotide sequence corresponding to the biological sample of the selected row.

Samples that are substantially similar can be highlighted with a second color (e.g., blue). A cluster can be defined by the rows (samples) that are visually identified. The cluster of biological samples can be identified based on a predefined threshold. In some instances, the predefined threshold indicates a number of SNPs, in which any biological sample having a number of SNPs below the predefined threshold can be clustered for the given biological sample. In some instances, the predefined threshold is modified through a user-interface element of the interactive dendrogram.

B. Configuration of Interactive Dendrogram

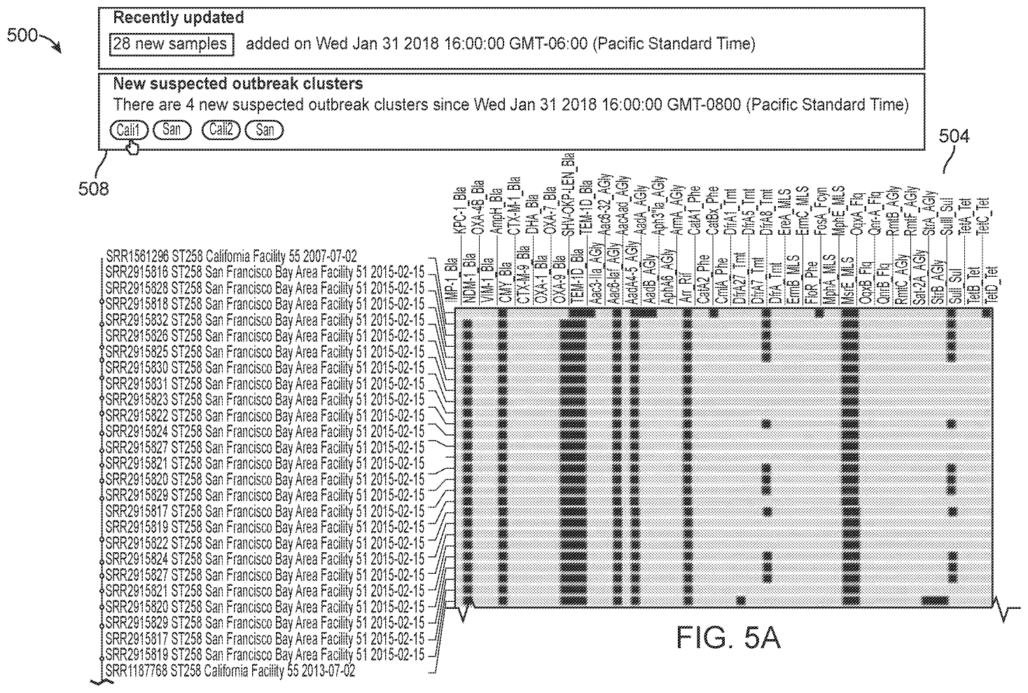

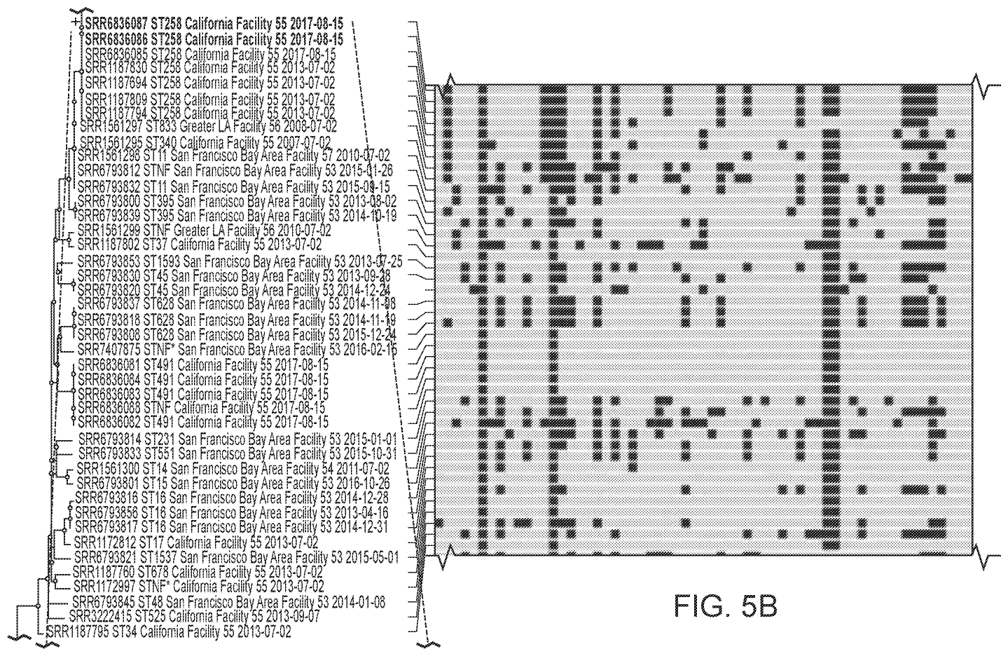

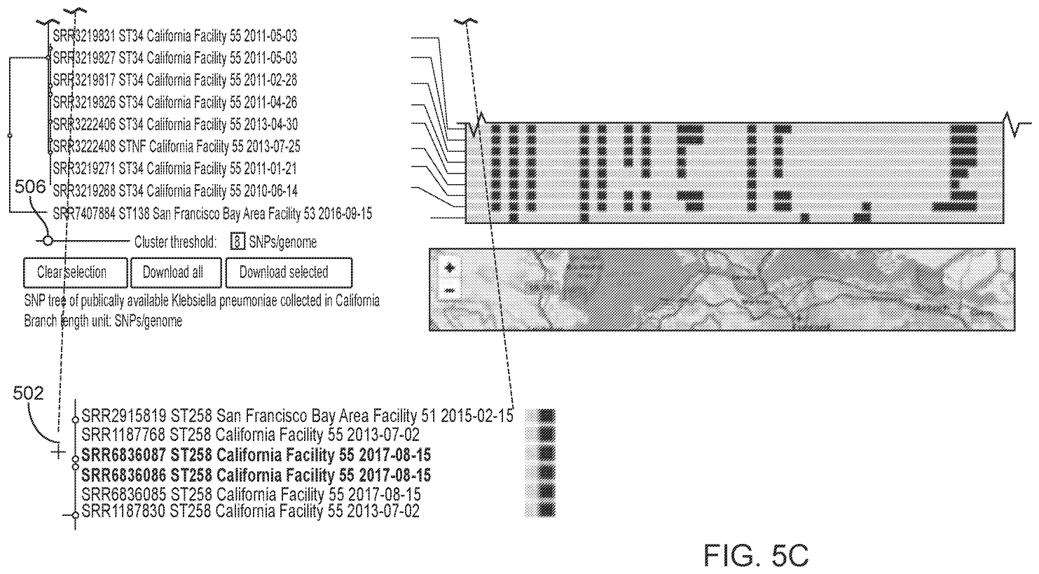

FIGS. 5A-C illustrate a first set of example screenshots of an interactive dendrogram 500 of a user interface in accordance with some embodiments. In some embodiments, the first set of example screenshots of FIGS. 5A-C are shown in the same screen of a user interface. The interactive dendrogram 500 may include the tree of rows 502 corresponding to biological samples collected from different regions and facilities. The tree of rows 502 can be presented on a left or right portion of the interactive dendrogram 500. In some instances, the interactive dendrogram 500 may display a tree of columns that is placed on its top portion, in which each column of the tree can represent a biological sample stored in the database server. Each row of the tree 502 may include information stored in the corresponding database record, including an identifier of a nucleotide sequence, an identifier of a subject, a name of a facility from which the biological sample was collected, and a date on which the biological sample was collected. The interactive dendrogram can be generated via various techniques, including tree generation methods described in Stamatakis et al. 2005 and phylogenetics-visualization methods described in Shank et al. 2018.

Each row of the tree 502 can be distributed on the tree 502 based on its sequence similarity relative to another sequence corresponding to another row. In particular, rows closer together may indicate that corresponding biological samples have similar nucleotide sequences. Conversely, rows far from each other may indicate that corresponding biological samples have different nucleotide sequences. A similarity metric may be used to indicate similarity of nucleotide sequences. The metric may include a number of SNPs between the sequences corresponding to the biological samples. To distribute the rows of the tree 502 in accordance with sequence similarities, a hierarchical clustering algorithm can be used to process the similarity metrics corresponding to the biological samples. In some instances, results outputted by the hierarchical clustering algorithm are used to identify a set of suspected outbreak clusters 508. Each cluster of the set of suspected outbreak clusters 508 may include biological samples that contribute to a suspected infectious disease outbreak, i.e., infected with an identical pathogen or different strains of the same pathogen. A selection of a cluster of the set of suspected outbreak clusters 508 can be highlighted or visually indicated with a color (“red”).

In some instances, each row of the tree 502 is connected to other rows through a plurality of branches. A branch between two rows of the tree 502 may indicate an extent of similarity between sequences of biological samples that correspond to the two rows. For example, a short branch between two rows may indicate that corresponding biological samples have substantially similar, if not identical, nucleotide sequences. A long branch, or a branch that traverses along other branches to reach the other row, may indicate that biological samples corresponding to two rows have significantly different nucleotide sequences.

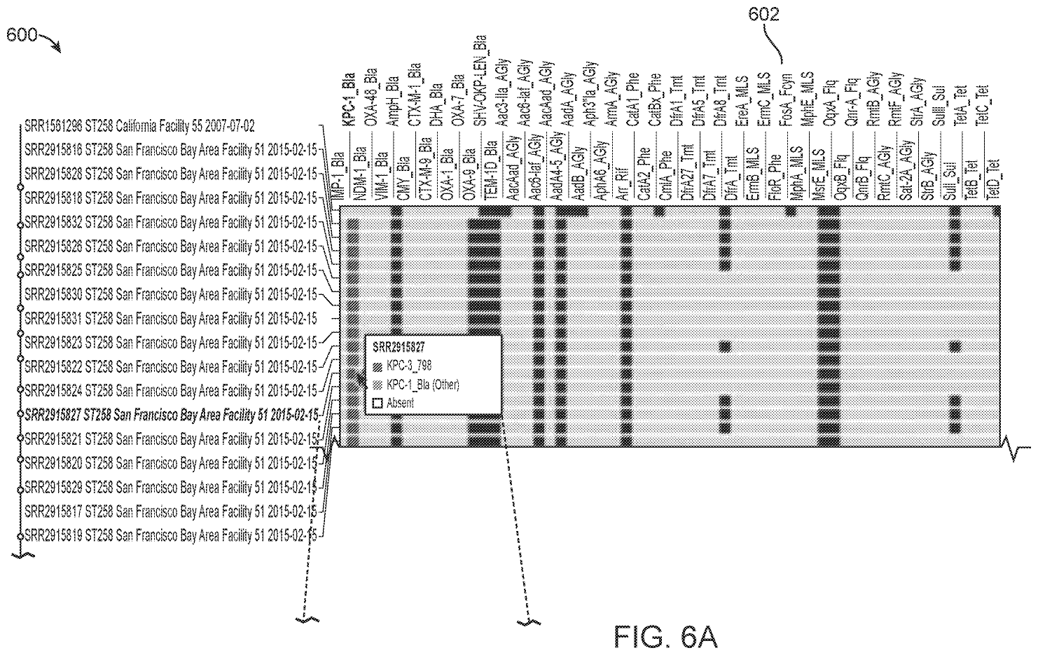

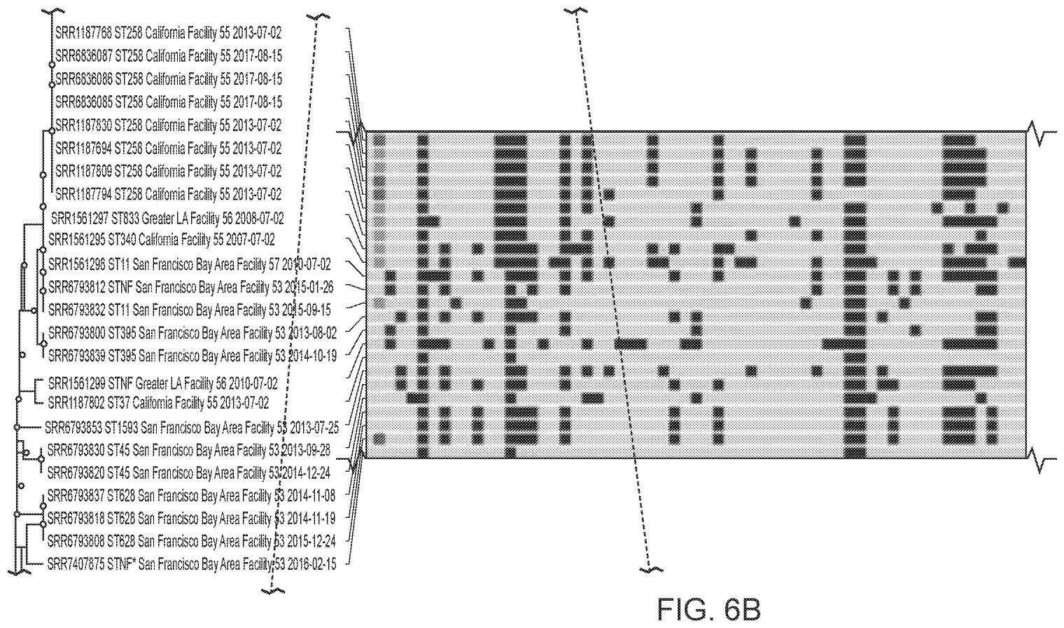

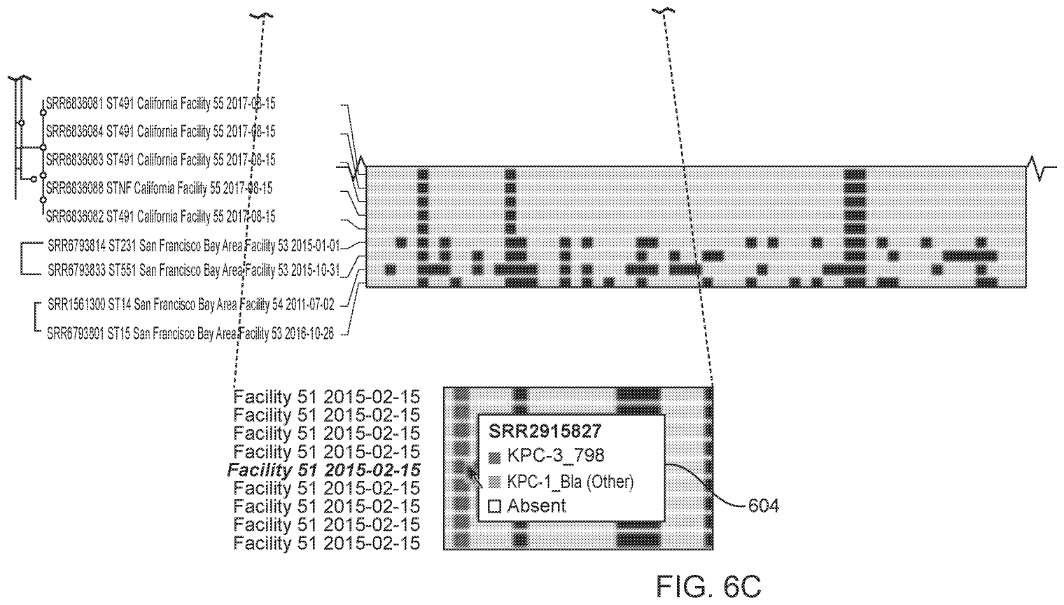

FIGS. 6A-C illustrate a second set of example screenshots of an interactive dendrogram 600 that provides a more detailed view of a heatmap in accordance with some embodiments. In some embodiments, the second set of example screenshots of FIGS. 6A-C are shown in the same screen of a user interface. A portion of the interactive dendrogram 600 may indicate a heatmap that provides a high-level view of genetic similarities between biological samples corresponding to the tree of rows. The heatmap may be formed based on sets of columns 602, in which each set of columns may correspond to a biological sample retrieved from the database server. In turn, a set of columns may include at least one column indicating whether a gene is present in the biological sample. To identify whether a particular gene is present in the biological sample, a sequence analyzer may determine whether a nucleotide sequence of the particular gene matches at least part of the nucleotide sequence the biological sample. In some instances, the gene identified by a column corresponds to an antibiotic-resistant gene.

The columns of the sets of columns 602 can be interactive. For example, in response to a selection (e.g., a click action, a hover action) of a particular column of a row, the interactive dendrogram 600 may display information 604, which may include a nucleotide-sequence identifier corresponding to selected column (e.g., SRR2916827), an indication of the gene being present (e.g., color), and a legend that indicates gene variants that correspond to the gene of the selected column. The legend indicated by the information 604 may include a first gene variant (e.g., KPC-3_798) labeled with a first color, a second gene variant (e.g., KPC-1_B1a) labeled with a second color, and so on. In this example, the first color associated with the column may indicate that the biological sample includes a part of the nucleotide sequence that corresponds to the KPC-3_798 gene variant.

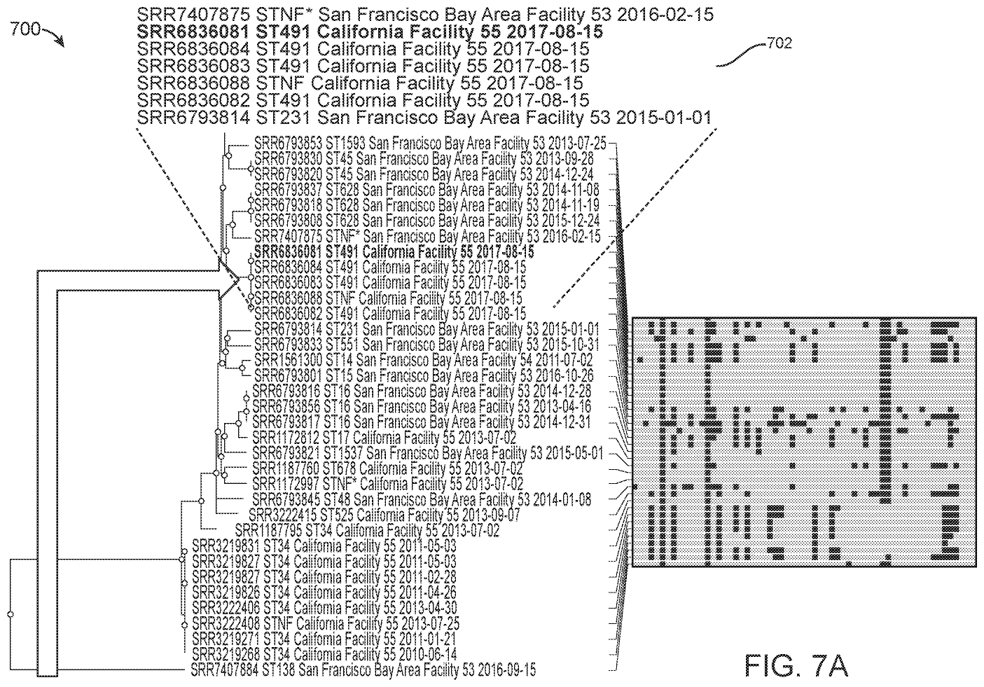

FIGS. 7A-B illustrate a third set of example screenshots of an interactive dendrogram 700 that identifies clusters of biological samples in accordance with some embodiments. In some embodiments, the third set of example screenshots of FIGS. 7A-B are shown in the same screen of a user interface. The interactive dendrogram 700 may receive a selection (e.g., a hover operation) of a biological sample. In response to the selection, the interactive dendrogram 700 may automatically identify a cluster 702 of biological samples that have similar nucleotide sequences to the sequence of the selected biological sample. The cluster 702 be highlighted or otherwise visually indicated to be distinct from other biological samples in the interactive dendrogram 700. In some instances, a selected biological sample may be highlighted in a first color (e.g., red), and additional samples in the cluster 702 may be highlighted in a second color (e.g., blue). Thus, cluster 702 can correspond to samples highlighted in either color.

The biological samples in the cluster 702 can be investigated through different aspects so as to detect an occurrence of an infectious disease outbreak. For example, a single pathogen can be identified from nucleotide sequences corresponding to the cluster of biological samples 702 that were collected from the same region at similar dates. The single pathogen may indicate an outbreak of an infectious disease in such same region. Identifying the cluster of biological samples 702 may lead to an efficient detection of infectious disease outbreaks, as opposed to solely relying on branches of a dendrogram.

To determine whether a biological sample should be added into the cluster 702 of biological samples, a number of SNPs can be counted between the biological sample and the selected biological sample. If the number of SNPs is under a predetermined threshold, the biological sample may be added into the cluster of biological samples 702. On the other hand, if the number of SNPs exceeds the predetermined threshold, the biological sample may be excluded or otherwise removed from the cluster of biological samples 702.

The predetermined threshold can indicate an upper limit of SNPs when determining similarity of nucleotide sequences between the two biological samples. The initial threshold can include a default number of SNPs. In some instances, some pathogens may have a different threshold of SNPs to be considered genetically similar (e.g., 3 SNPs vs. 20 SNPs). The predetermined threshold can be modified based on adjusting an interactive user-interface element 704 of the interactive dendrogram 700. For example, the interactive user-interface element 704 can include a range slider and/or a text box. Modifying the predetermined threshold may result in automatically identifying a different cluster of biological samples. For example, modifying the predetermined threshold to a greater value (e.g., 20 SNPs) can result in an expanded cluster of biological samples relative to the cluster 702 identified at initial threshold (e.g., 10 SNPs). In another example, modifying the predetermined threshold to a lesser value (e.g., 3 SNPs) can result in a smaller cluster of biological samples relative to the cluster 702 identified at initial threshold (e.g., 10 SNPs).

C. Method for Interactive Dendrogram

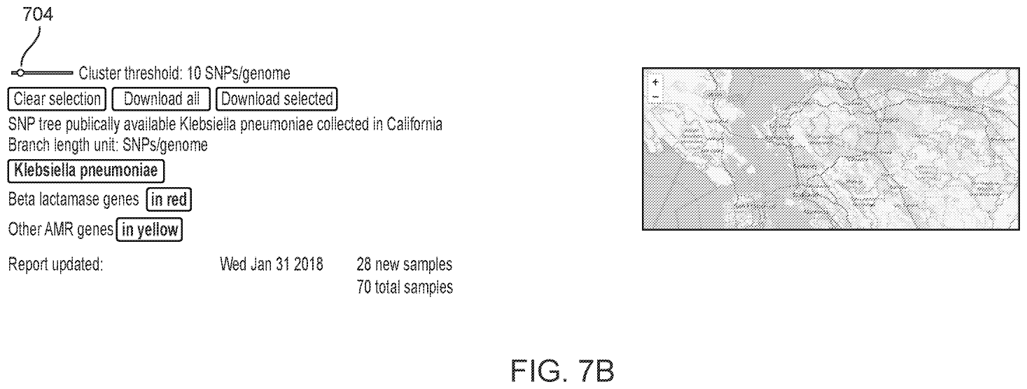

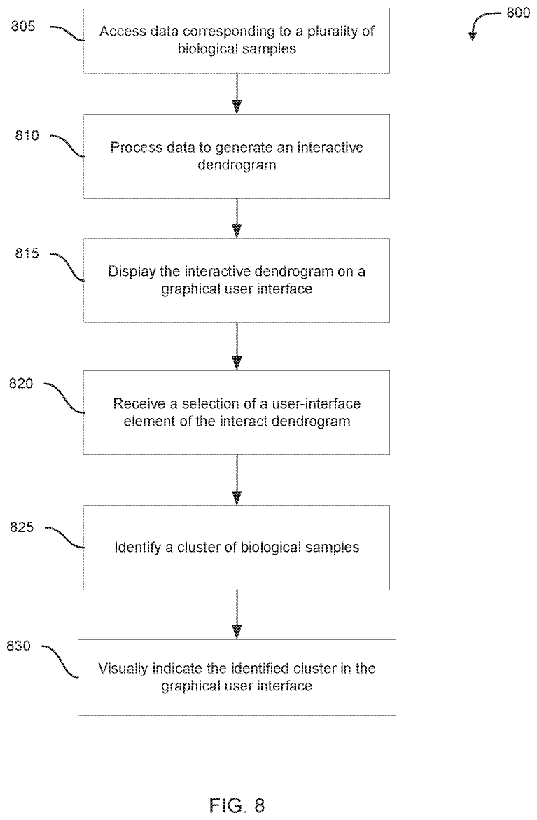

FIG. 8 illustrates a process 800 in which a cluster of biological samples is identified from an interactive dendrogram in accordance with some embodiments. Process 800 may be performed by the interactive dendrogram component 110 of FIG. 1. A web interface (for example) can be provided to a client device (e.g., the client device 102 of FIG. 1), on which the interactive dendrogram can be displayed.

At step 805, data corresponding to a plurality of biological samples can be accessed. The data may include a nucleotide sequence for each of the plurality of biological samples retrieved from a database server. In some instances, each biological sample in the data may be associated with a plurality of genes, in which each gene of the plurality of genes is identified based on at least part of the nucleotide sequence (e.g., nucleotide sub-sequence). The data may additionally indicate degrees of similarities between nucleotide sequences of the biological samples.

At step 810, the data can be processed to generate an interactive dendrogram (e.g., the interactive dendrogram 700 of FIG. 7) that includes an interactive portion that depicts a set of user-interface elements. Each user-interface element may represent a biological sample of the plurality of biological samples. In addition, the user-interface elements of the interactive dendrogram are arranged within the interactive portion based on the degree of similarity of the sequences of the biological samples that are respectively represented by the set of user-interface elements.

At step 815, the interactive dendrogram can be displayed on a graphical user interface. In some instances, a heatmap corresponding to the biological samples is displayed in another portion of the graphical user interface. The heatmap may include a set of columns corresponding to each biological sample in the interactive dendrogram. A column of the set of columns may indicate whether a gene is present in the corresponding biological sample. To identify whether a particular gene is present in the biological sample, a sequence analyzer may determine whether a nucleotide sequence of the particular gene matches at least part of the nucleotide sequence the biological sample.

At step 820, a user-interface element of the interact dendrogram can be selected through the interactive portion of the graphical user interface. For example, the user-interface element can be selected (e.g., click, hover) by a user of the graphical user interface.

At step 825, a cluster of biological samples can be identified in response to the selection. A biological sample can be included in the cluster by determining whether a number of SNPs between a sequence of the biological sample and a biological sample corresponding to the selected user-interface element is under a threshold. The threshold may be a number of SNPs that indicate an extent of variations between nucleotide sequences of corresponding to two given biological samples. In some instances, the cluster of biological samples can be highlighted. For example, the selected biological sample can be visually indicated in a first color (red) and genetically-related biological samples in the cluster can be visually indicated in a second color (blue).

At step 830, the identified cluster of biological samples can be visually indicated in the graphical user interface. In some instances, the threshold is updated using a text box or a range-slider of another portion of the interactive dendrogram. For example, in response to receiving a value greater than the value corresponding to the initial threshold, the threshold can be updated such that a larger cluster of biological samples can be identified. In another example, in response to receiving a value lesser than the value corresponding to the initial threshold, the threshold can be updated such that a smaller cluster of biological samples can be identified.

### IV. SIMILARITY MATRIX

A. Overview

A similarity matrix may be provided in the user-interface that can be used to identify a degree of similarity between sequences corresponding to two or more biological samples. The similarity matrix may correspond to different sets of samples selected from an interactive dendrogram (e.g., the interactive dendrogram 500 of FIGS. 5A-C), in order to provide insight with respect to the similarity of sequences corresponding different samples. For example, a genetic-variation metric identified by the similarity matrix may reveal that two biological samples collected from different regions may have been infected with the same pathogen.

The similarity matrix may include rows and columns corresponding to samples selected from the interactive dendrogram. The similarity matrix can be a symmetric matrix, in which a first row and column corresponds to a first biological sample, a second row and column corresponds to a second biological sample, and so on. The similarity matrix may include a plurality of matrix elements corresponding to a row and a column of the similarity matrix. Each matrix element of the plurality may indicate the genetic-variation metric (e.g., a number of SNPs) between two samples of a corresponding row and column. The genetic-variation metric may indicate a degree of similarity between sequences of the two corresponding biological samples.

To generate the similarity matrix, a database can be configured such that the genetic-variation metrics corresponding to the matrix elements are pre-computed. In particular, the genetic-variation metrics can be pre-computed for all samples listed in the interactive dendrogram. The pre-computed metrics can be stored in a database table in a database server (e.g., the database server 106 of FIG. 1). As individual biological samples are selected from the interactive dendrogram, matrix elements of the similarity matrix can be added and populated with the genetic-variation metrics retrieved from the database table. The similarity matrix may thus display a sub-table that provides the genetic-variation metrics corresponding to the selected biological samples. In some instances, the sub-table is downloaded and locally stored in a client device.

For each biological sample selected through the interactive dendrogram, matrix elements can be automatically added into the similarity matrix such that dimensions corresponding the similarity matrix can increase by 1. For example, when two biological samples are selected from the interactive dendrogram, the similarity matrix with 2×2 dimensions may be automatically generated. Additional biological samples can be selected after the initial similarity matrix is generated. When an additional biological sample is selected, matrix elements can be added to the similarity matrix such that the dimensions of the similarity matrix can increase in proportion to the number of the additional biological samples. For example, a similarity matrix with 3×3 dimensions can increase to 5×5 dimensions in response to two additional biological samples being selected from the interactive dendrogram. Conversely, biological samples in the similarity matrix can be unselected, at which matrix elements can be removed from the similarity matrix such that the dimensions of the similarity matrix can decrease in proportion to the number of biological samples that were unselected. In some instances, the similarity matrix generates an average value of SNPs among the biological samples of the similarity matrix (for example) and a number of SNPs that corresponds to an unselected biological sample that has the most similar sequence to those of the biological samples of the similarity matrix.

B. Configuration of Similarity Matrix

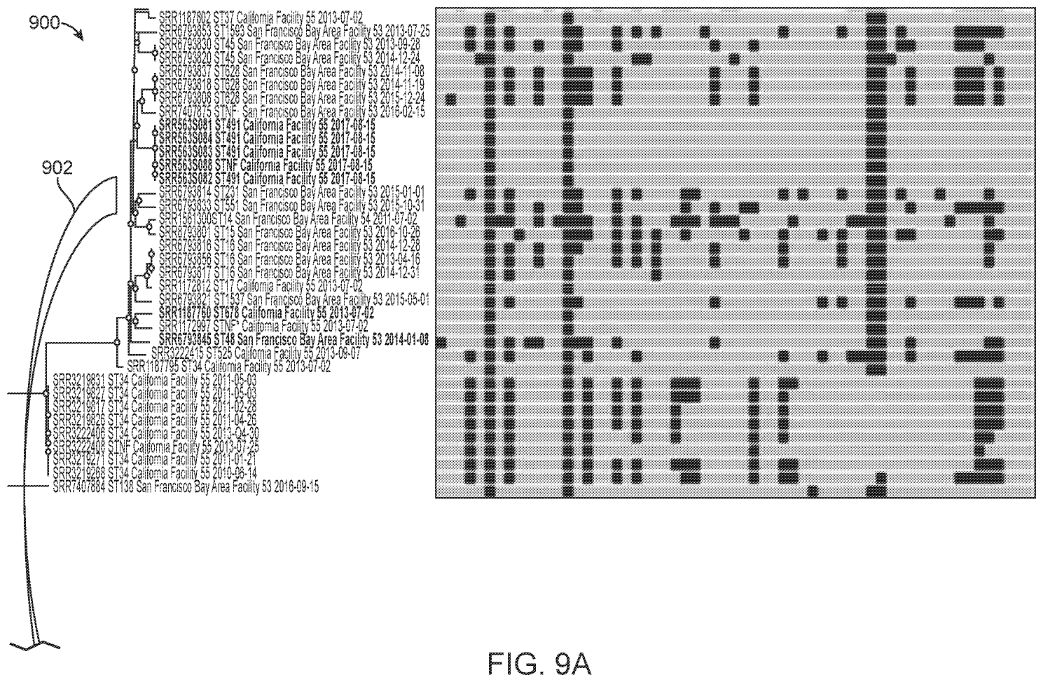

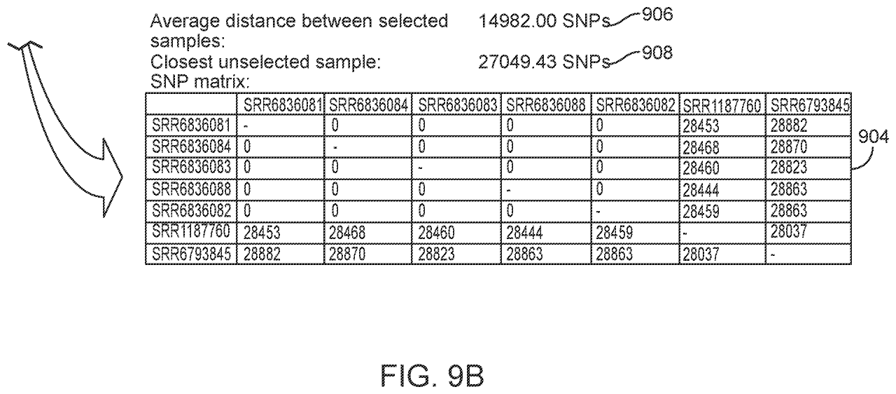

FIGS. 9A-B illustrate a set of example screenshots of an interactive dendrogram and a similarity matrix 900 in accordance with some embodiments. In some embodiments, the set of example screenshots of FIGS. 9A-B are shown in the same screen of a user interface. To determine whether biological samples are related to a single pathogen, the biological samples can be selected from an interactive dendrogram (e.g., the interactive dendrogram 500 of FIGS. 5A-C). In response, the similarity matrix can be generated, in which the similarity matrix may include genetic-variation metrics between sequences of the selected biological samples. The genetic-variation metrics may indicate a degree of similarity between sequences of the selected biological samples. The degree of similarity may include a number of SNPs. The genetic-variation metrics can be used with other information corresponding to the selected biological samples (e.g., location and date on which the selected biological samples were collected) to detect whether two biological sample refer to the similar pathogens capable of causing an infectious disease outbreak.

The similarity matrix can be generated by selecting at least one biological sample from the interactive dendrogram. For example, a set of biological samples can be selected from an interactive dendrogram 902. The selection can occur based on various types of user-interface actions, including a click operation, a shift-click operation, and a control-click operation. As the set of biological samples are selected, a similarity matrix 904 can be generated. In some instances, rows and columns of the similarity matrix 904 are automatically added as each biological sample is selected from the interactive dendrogram 902. Matrix elements corresponding to the added rows and columns can be populated with data that corresponds to a genetic-variation metric (e.g., a number of SNPs). For example, a row corresponding to a first biological sample and a column corresponding to a second biological sample may generate a matrix element that indicates the genetic-variation metric between sequences corresponding to the first and second biological samples. In addition, rows and columns (and corresponding matrix elements) of the similarity matrix 904 can be automatically removed as each biological sample is unselected from the interactive dendrogram 902. By analyzing the genetic-variation metrics of selected biological samples, information correlating to the epidemiological information of pathogens can be discovered.

Additional information corresponding to selected biological samples can be presented with the similarity matrix 904. For example, an average metric 906 can be calculated and presented on the user interface. The average metric 906 may include an average value corresponding to the numbers of SNPs generated by the similarity matrix 904. In another example, a closest-sequence metric 908 can be calculated and presented on the user interface. The closest-sequence metric 908 may indicate the number of SNPs corresponding to an unselected biological sample that has the most similar sequence to those of the biological samples in the similarity matrix 904. The average metric 906 and/or the closest-sequence metric 908 can be compared to determine whether the biological samples in the similarity matrix 904 are genetically similar. For example, if the average metric 906 indicates a lower value (e.g., 15000 SNPs) as compared to the closest-sequence metric 908 (e.g., 27000 SNPs), it can be determined that the biological samples in the similarity matrix 904 are genetically similar. Conversely, if the average metric 906 indicates a higher value (e.g., 28000 SNPs) as compared to the closest-sequence metric 908 (e.g., 23000 SNPs), it can be determined that the biological samples in the similarity matrix 904 are genetically different. The additional information may thus provide another insight in discovering epidemiological information corresponding to pathogens.

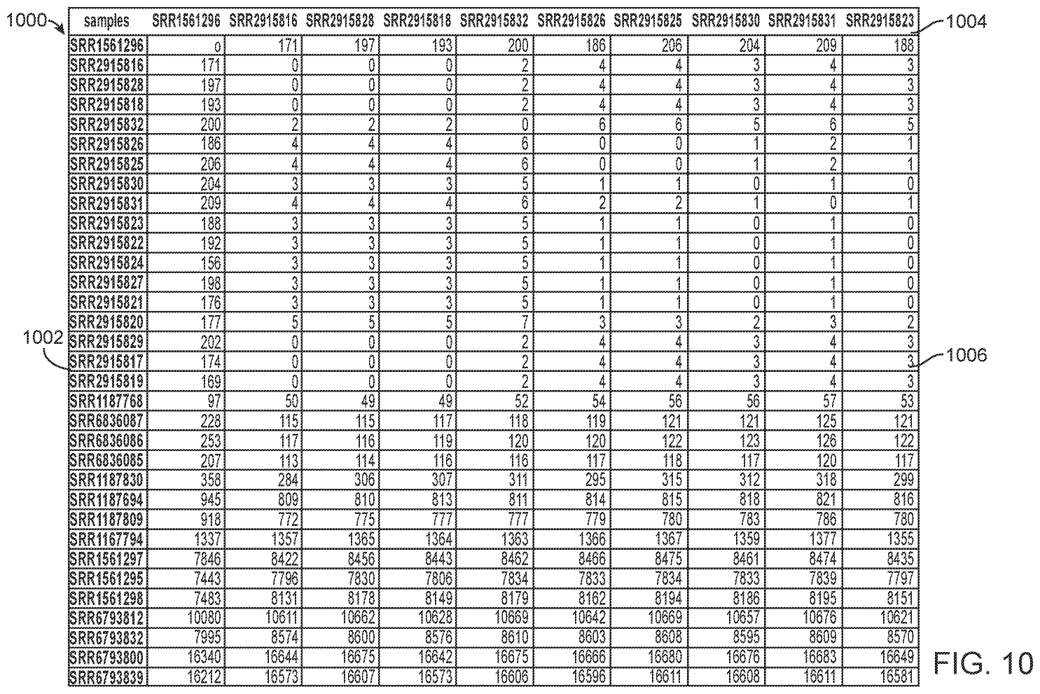

FIG. 10 illustrates an example database 1000 for generating a similarity matrix in accordance with some embodiments. As described herein, matrix elements of a similarity matrix can be added as a biological sample is selected form the interactive dendrogram. Genetic-variations metrics corresponding to the matrix elements can be pre-computed and stored in the database 1000 to increase a rate of data retrieval during user-interface interactions. The database 1000 can be a larger version of the similarity matrix (e.g., the similarity matrix 900 of FIGS. 9A-B), in which genetic-variation metrics corresponding to each and every biological sample in an interactive dendrogram can be stored. With respect to pre-computation of the genetic-variation metrics, the genetic-variation metrics can be calculated as the biological samples and corresponding nucleotide sequences are uploaded in a database server.

As the biological samples are selected from the interactive dendrogram to be presented in the similarity matrix, a database query can be constructed to retrieve genetic-variation metrics which can be populate the corresponding matrix elements. For example, a row 1002 may correspond to a biological sample with sequence SRR118779 and a column 1004 may corresponds to a biological sample with sequence SRR2915823. As biological samples with sequences corresponding to SRR118779 and SRR2915823 are selected, a genetic-variation metric 1006 having a value of 1355 SNPs can be selected. The 1355 SNPs may be associated with a matrix element of the similarity matrix that corresponding to the sequences SRR118779 and SRR2915823.

C. Method for Similarity Matrix

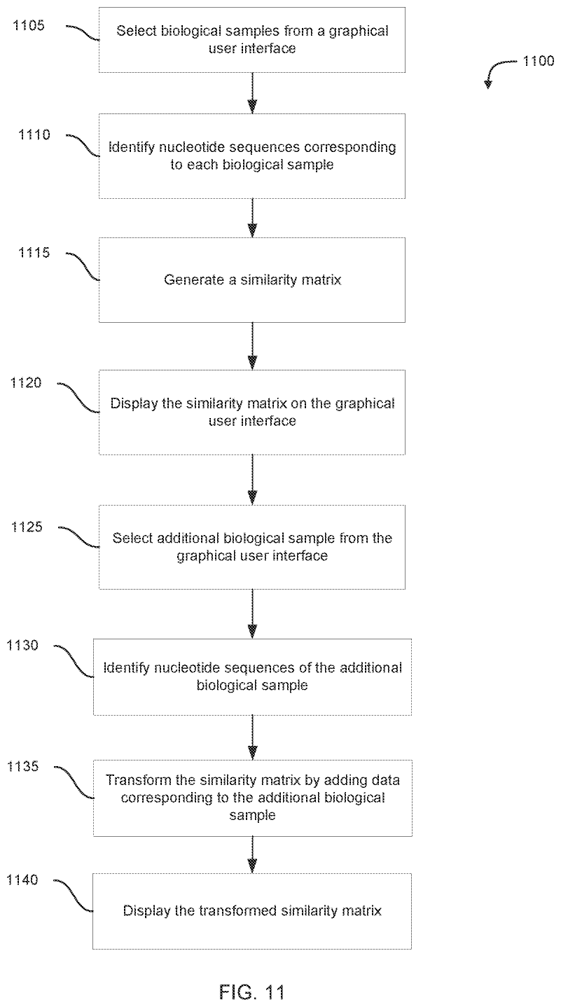

FIG. 11 illustrates a process 1100 for configuring a similarity matrix in accordance with some embodiments. Process 1100 may be performed by the similarity interface 114 of FIG. 1. The similarity matrix can be concurrently displayed with an interactive dendrogram, in which the interactive dendrogram can be displayed in a first area of the graphical user interface while the similarity matrix can be displayed on a second area of the graphical user interface.

At step 1105, two biological samples can be selected from a graphical user interface. In some instances, the two biological samples are selected by interacting with a portion of an interactive dendrogram. Alternatively or additionally, one of the two biological samples can be selected by determining that variation between nucleotide sequences of biological sample and another biological sample of the two biological samples is within a predetermined single-nucleotide-polymorphism (SNP) threshold. One of the two biological sample may also be selected based on an indication that both biological samples belong a suspected outbreak cluster (e.g., the suspected outbreak cluster 508 of FIGS. 5A-C).

At step 1110, nucleotide sequences corresponding to each of the two biological samples can be identified. The sequences can be identified by accessing database records stored in a database that correspond to the selected biological samples. The database may store data relating to a plurality of biological sample, in which the data may include pre-computed values. Each pre-computed values may indicate a number of variations between nucleotide sequences of any given two biological samples stored in the database.

At step 1115, a similarity matrix can be generated. The similarity matrix may include a matrix element for each of the two selected biological samples, and the matrix element may indicate a number of variations between the identified nucleotide sequences of the biological samples. The number of variations may refer to a number of SNPs between the nucleotide sequences. In some instances, the number of variations may be a pre-computed value that is retrieved from the database.

At step 1120, the similarity matrix can be displayed on the graphical user interface. The similarity matrix may include rows and columns, in which each row and column indicates a selected biological sample. The matrix elements corresponding to the two biological samples can be added to a corresponding row and column of the similarity matrix. For example, a row corresponding to a first biological sample and a column corresponding to a second biological sample may include a matrix element that indicates the number of SNPs between nucleotide sequences corresponding to the first and second biological samples.

At step 1125, a selection of an additional biological sample can be received while the similarity matrix is being displayed. In some instances, the additional biological sample is automatically selected based on a determination that variation between nucleotide sequences of the additional biological sample and at least one of the two biological samples in the similarity matrix is within a predetermined single-nucleotide-polymorphism (SNP) threshold.

At step 1130, a nucleotide sequence corresponding to the additional biological sample can be identified. Similar to above, the sequence can be identified by accessing database records that correspond to the additional biological sample. In some instances, as the additional biological sample is selected, a database query can be constructed and submitted to retrieve a number of variations between nucleotide sequences between the additional biological sample and one of the two biological samples represented in the similarity matrix.

At step 1135, the similarity matrix can be transformed by adding matrix elements that correspond to the additional biological sample. A number of added matrix elements can be proportional to the number of biological samples in the similarity matrix. For example, the number of added matrix elements can be one less than twice the number of biological samples in the similarity matrix. Further, each added matrix element may indicate a number of variations between the nucleotide sequences of the additional biological sample and a given biological sample in the similarity matrix.

At step 1140, the transformed similarity matrix can be displayed on the graphical user interface. In some instances, one of the three biological samples is selected in the similarity matrix. In response to the selection, the transformed similarity matrix can be transformed again by removing the matrix elements that correspond to the selected biological sample.

### V. DIFFERENTIAL ACCESS

A. Overview

The user-interface may provide a comprehensive view of pathogen presence based on analyzing sequences of biological samples collected from several facilities and regions. Such comprehensive view may allow detection of infectious disease outbreaks across several regions and contain the infectious disease outbreaks for further transmission. To prevent sensitive information (e.g., name of the facility, consortium) from being shared by unauthorized users, access to data corresponding to one or more categories can be restricted. Extent of the restriction can be determined (for example) based on comparing a user-affiliated group to a group associated with an entity that uploaded the data, a user-affiliated consortium to a consortium associated with the entity that uploaded the data, a user-affiliated region to a region associated with the entity that uploaded the data, and so on.

For example, if an accessing user is affiliated the group that matches the group of an entity that uploaded the biological samples (e.g., SF), the database server may provide the biological samples and corresponding nucleotide sequences without any restriction. In another example, if the accessing user is affiliated with a different group (e.g., OAK) but the same consortium as those of the entity that uploaded the biological samples (e.g., Alameda County), the database server may restrict information that could reveal the group name of the entity. Based on the extent of the restriction, the database server can redact the information from the user interface (for example) or replace the information with other generic information (for example). Referring to the other example above, the user-interface can replace “SF” with a generic identifier such as “Group 22.”

B. Example Configuration for Differential Access

To access information in the user-interface including an interactive dendrogram corresponding the biological samples, a user may be registered and provided an account. The registration may include receiving, from the user, information indicating a group associated with the user and a consortium associated with the group. In some instances, the consortium is automatically identified based on the group associated with the user. For example, a group may identify a healthcare facility in a region which corresponds to a consortium of healthcare facilities in the same region.

Differential access of the database can be configured by associating or “tagging” each database record with a group that uploaded the files that correspond to the biological samples stored in the database record. The group association may be used to determine how information retrieved from the database record will be redacted. In addition, each column of the database can be marked with different levels of access. For example, the levels of access may include “owner,” “consortium,” and “public.” A “public” level of access may indicate information that can be derived from other public sources. Based on the different level of access, a database server may dynamically redact information corresponding to each column based on a comparison between a user's group affiliation (for example) and the column's access level. In some instances, additional security measures can be provided by restricting data corresponding to unmarked columns from being transmitted to the user interface. Table 1 provides an example set of access rules for each level of access:

TABLE 1

| | and the column | |
|---|---|---|
| If the user . . . | is marked, | then take this action |
| belongs to the group that | “owner” | transmit this column for |
| owns this record | “consortium” | this record. |
| | “public” | |
| does not belong to the group | “owner” | do not transmit this |
| that owns this record, but | | column. |
| belongs to a group in the | “consortium” | transmit this column for |
| same consortium as the group | “public” | this record |
| that owns the record | | |
| does not belong to the group | “owner” | do not transmit this |
| that owns this record, and | “consortium” | column for this record. |
| does not belong to the same | “public” | transmit this column for |
| consortium as the group that | | this record. |
| owns this record | | |

In some instances, each of the different levels of access for each column of the database is defined by a set of rules. The set of rules can be configured by using object relational mapper (ORM) classes that correspond to the database. For example, an ORM class may correspond to database records storing biological samples uploaded by a particular group. The ORM class in this example can include program code that specifies access levels for each column ofthe database records. In particular, for each access level specified in the ORM class (e.g., consortium_attrs), one or more columns of_the database records can be associated (e.g., array values). Example 1 provides an example program code to configure access levels of the columns corresponding to the database records:

### Example 1

```
class Sample(DeclarativeBase, SerializeMixin):
  _{——}tablename_{——} = “samples”
  _{——}public_attrs_{——} = [“id”, “genus”, “species”,
“sequence_type”, “genes’, “processing_status”]
  _{——}consortium_attrs_{——} = [“patient_deidentifier”, “facility”,
“specimen_collection_date”, “sample_upload_date”,
“specimen_source”, “owning_group”, “processing_status”,
“genes”]
  _{——}owner_attrs_{——} = [“internal_id”, “fastq_filename”,
“upload_user”]
```

An example use-case scenario is presented to illustrate differential access of information stored in the database server. A database record that includes biological sample collected from “County Health Department A” is stored in the database server. At least part of the database record is shown as follows:

| id | species | facility | specimen_source | internal_id | upload_user |
|---|---|---|---|---|---|
| 47 | Pneumoniae | St. John's | Blood | COUNTY_A_03 | elizabeth@county-a.gov |
| | | Hospital | | | |

A first user may request to access data corresponding to the database record. For example, the first user may desire to verify a region associated with the database record to determine whether a biological sample corresponding to the database record if from the same region of another biological sample shown in the interactive dendrogram. In response, the database server may use registration information of the first user to identify a first group. The first group may then be compared to a group associated with the database record (i.e., County Health Department A). In this use-case scenario, the first group may indicate “County Health Department A”, which matches the group corresponding to the database record. As a result of determining that the groups match, the database server may transmit all information stored in the database record without any redactions.

A second user may also request to access data corresponding to the database record. The database server may use registration information of the second user to identify a second group named “County Health Department B.” The database server may determine that the second group and the group corresponding to the database record (i.e., County Health Department A) do not match. As a result, the database server may make another determination whether the groups belong to the same consortium. In this use-case scenario, the groups belong to the same consortium, at which the database server can transmit a partially-redacted database record under the following access rules: (i) information from all columns having public access level are transmitted; (ii) information from all columns having consortium access level are transmitted; and (iii) none of the information from columns having owner access level is transmitted. The partially-redacted database record to be transmitted to the second user is presented as follows:

| id | species | facility | specimen_source |
|---|---|---|---|
| 47 | Pneumoniae | St. John's | Blood |
| | | Hospital | |

A third user may request to access data corresponding to the database record. The database server may use registration information of the third user to identify a third group named “County Health Department N.” The database server may determine that the third group and the group corresponding to the database record do not match (i.e., County Health Department A). Further, the database server may determine that the groups belong to different consortiums. As a result, the database server can transmit a fully-redacted database record under the following access rules: (i) information from all columns having public access level are transmitted; (ii) none of the information from columns having consortium access level are transmitted; and (iii) none of the information from columns having owner access level is transmitted. The fully-redacted database record to be transmitted to the third user is presented as follows:

| id | species |
|---|---|
| 47 | Pneumoniae |

As presented by the above use-case scenario, the database server may redact sensitive information before data is transmitted to the user-interface. Biological samples presented in the interactive dendrogram (for example) can be formatted based on the redacted database records. For example, if a database record is a partially-redacted record, the user-interface may display a biological-sample identifier that includes a name of the biological sample (“Pneumoniae”) and a name of the facility (“St. John's Hospital”), but an unknown date on which the biological sample was collected (“unknown”). In effect, the user may still access comprehensive data of biological samples collected from various regions and analyze such data to detect infectious disease outbreaks, while avoiding access to sensitive data.

C. Method for Differential Access

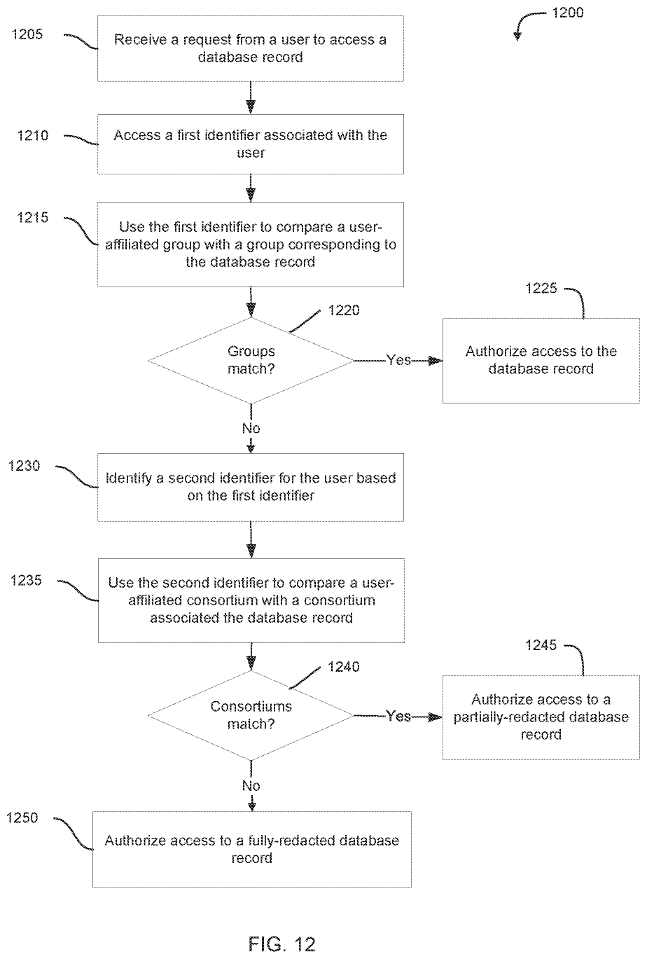

FIG. 12 illustrates a process 1200 for restricting access to biological-sample data in accordance with some embodiments. The process 1200 may be performed by the access controls component 107 of the database server 106 in FIG. 1. While allowing relevant information to be displayed for pathogen analysis, access to data corresponding to one or more categories can be restricted to prevent sensitive information (e.g., name of the facility, consortium) from being shared by unauthorized users.

At step 1205, when a user requests access to a database record corresponding to biological sample. The database record may be stored in a database and can include data corresponding to a biological sample collected from a geographical region. The data may also be processed to identify a nucleotide sequence corresponding to the biological sample, which can be stored in the database record. The data of the database record may be uploaded by a group of users authorized to access the database storing the database record. The database may include database records corresponding to a plurality of biological samples and nucleotide sequences corresponding to each of the plurality of biological samples.

At step 1210, a first identifier associated with the user can be retrieved. For example, the first identifier of the user may identify a group or a facility with which the user is affiliated. In some instances, the first group identifier of the user is identified based on registration information corresponding to the user.

At step 1215, the first identifier of the user can be used to compare the user-affiliated group with the group that uploaded the data corresponding to the database record and authorized to access the database. The group of users that uploaded the data can be a facility that collected and uploaded biological-sample data corresponding to the database record.

At step 1225, access to the database record can be authorized if it is determined that the user-affiliated group and the authorized group match (“Yes” branch of step 1220). The database record may include all of the information stored in the database record, including a subject identifier and a date on which the biological sample was collected. As such, full access to the database record is granted.

At step 1230, a second identifier can be identified for the user in the event that the user-affiliated group and the authorized group do not match (“No” branch of step 1220). The second identifier may indicate a collection of groups (e.g., a consortium) to which the user-affiliated group corresponds. The user-affiliated collection may be identified based on a geographic region corresponding to the user-affiliated group.

At step 1235, the second identifier can be used to compared the user-affiliated collection with a collection of groups corresponding to the authorized group. The collection corresponding to the authorized group may be identified based on a geographic region associated with the authorized group In some instances, the collection of the authorized group indicates a consortium of facilities (e.g., hospitals) that are located within the same geographic region (e.g., Alameda County).

At step 1245, access to a partially-redacted database record can be authorized if it is determined that the collections of groups match (“Yes” branch of step 1240). The partially-redacted database record may include data corresponding to a subset of columns of the database record. In some instances, redacting the part of the new database record includes replacing the part of the new database record with information that prevents disclosure of the first part of the new database record Accordingly, the partially-redacted record may include one or more anonymized parts.

At step 1250, access to a fully-redacted database record can be authorized in the event that the collections of groups do not match (“No” branch of step 1240). The fully-redacted database record may include data corresponding to a more restricted subset of columns of the database record in relation to the data of the partially-redacted database record.

### VI. COMPUTER SYSTEM

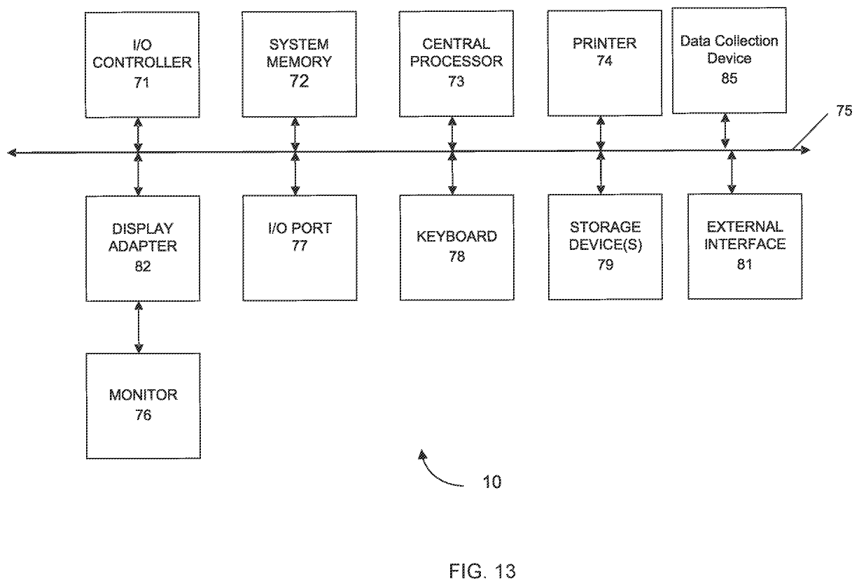

Any of the computer systems mentioned herein may utilize any suitable number of subsystems. Examples of such subsystems are shown in FIG. 13 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 13 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art.

### VII. REFERENCES

1. Center for Genomic Pathogen Surveillance (CGPS), PathogenWatch, 2018, https://pathogen.watch.
2. James Hadfield, Colin Megill, Sidney M Bell, John Huddleston, Barney Potter, Charlton Callender, Pavel Sagulenko, Trevor Bedford, Richard A Neher, Nextstrain: real-time tracking of pathogen evolution, Bioinformatics, Volume 34, Issue 23, 1 Dec. 2018, Pages 4121-4123, https://doi.org/10.1093/bioinformatics/bty407.
3. Gardy, J., Loman, N. Towards a genomics-informed, real-time, global pathogen surveillance system. Nat Rev Genet 19, 9-20 (2018). https://doi.org/10.1038/nrg.2017.88.
4. Harris S R, Feil E J, Holden M T, Quail M A, Nickerson E K, Chantratita N, Gardete S, Tavares A, Day N, Lindsay J A, Edgeworth J D, de Lencastre H, Parkhill J, Peacock S J, Bentley S D. Evolution of MRSA during hospital transmission and intercontinental spread. Science. 2010 Jan. 22; 327(5964):469-74. doi: 10.1126/science.1182395. PMID: 20093474; PMCID: PMC2821690.
5. Heng Li, Bob Handsaker, Alec Wysoker, Tim Fennell, Jue Ruan, Nils Homer, Gabor Marth, Goncalo Abecasis, Richard Durbin, 1000 Genome Project Data Processing Subgroup, The Sequence Alignment/Map format and SAMtools, Bioinformatics, Volume 25, Issue 16, 15 Aug. 2009, Pages 2078-2079, https://doi.org/10.1093/bioinformatics/btp352.
6. Li, H. (2018). Minimap2: pairwise alignment for nucleotide sequences. Bioinformatics, 34:3094-3100. doi:10.1093/bioinformatics/bty191.
7. Inouye, M., Dashnow, H., Raven, L. et al. SRST2: Rapid genomic surveillance for public health and hospital microbiology labs. Genome Med 6, 90 (2014). https://doi.org/10.1186/s13073-014-0090-6.
8. Shank, S., Weaver, S. & Kosakovsky Pond, S. phylotree.js—a JavaScript library for application development and interactive data visualization in phylogenetics. BMC Bioinformatics 19, 276 (2018). https://doi.org/10.1186/s12859-018-2283-2.
9. A. Stamatakis, T. Ludwig, H. Meier, RAxML-III: a fast program for maximum likelihood-based inference of large phylogenetic trees, Bioinformatics, Volume 21, Issue 4, 15 Feb. 2005, Pages 456-463, https://doi.org/10.1093/bioinformatics/bti191.

1. A computer-implemented method of uploading data to facilitate pathogen analysis of biological samples, the method comprising:
receiving, via a first area of a graphical user interface, a first file that includes a plurality of sample identifiers and a plurality of sequence-file identifiers, wherein each of the plurality of sample identifiers corresponds to a biological sample and a sequence-file identifier of the plurality of sequence-file identifiers;
receiving, via a second area of the graphical user interface, a set of nucleotide-sequence files, wherein each nucleotide-sequence file of the set of nucleotide-sequence files includes a nucleotide sequence and is associated with a sequence-file identifier;
determining that a first sequence-file identifier corresponding to a first sample identifier of the first file matches a second sequence-file identifier corresponding to a first nucleotide-sequence file of the set of nucleotide-sequence files, the first sample identifier corresponding to a first biological sample;
in response to the determining that the first sequence-file identifier matches the second sequence-file identifier, associating the first sample identifier to the nucleotide sequence stored in the first nucleotide-sequence file;
generating a first database record corresponding to the first biological sample, the first database record including the first sample identifier and the associated nucleotide sequence; and
storing the first database record into a database comprising information corresponding to a plurality of biological samples.

2. The computer-implemented method of claim 1, further comprising, in response to a user input, causing the graphical user interface to display the first database record.

3. The computer-implemented method of claim 1, wherein the first file and the set of nucleotide-sequence files are received via a drag-and-drop action performed via the graphical user interface.

4. The computer-implemented method of claim 1, further comprising:
causing the graphical user interface to display the plurality of sample identifiers of the first file in the first area of the graphical user interface prior to storing the first database record; and
in response to receiving a user authorization after displaying the plurality of sample identifiers of the first file, storing the first database record in the database.

5. The computer-implemented method of claim 1, further comprising:
determining that the set of nucleotide-sequence files do not include any nucleotide-sequence file having a sequence-file identifier that matches the first sequence-file identifier; and
in response to determining that the set of nucleotide-sequence files do not include any nucleotide-sequence file having a sequence-file identifier that matches the first sequence-file identifier, causing the graphical user interface to display an error message.

6. The computer-implemented method of claim 1, further comprising:
identifying one or more database records stored in the database, the one or more database records identified based on determining that biological samples corresponding to the one or more database records corresponds to a same pathogen; and
causing the graphical user interface to visually indicate that biological samples corresponding to the identified one or more database records relate to a suspected infectious disease outbreak.

7. A computer-implemented method of generating an interactive dendrogram that facilitates pathogen analysis of biological samples, the method comprising:
accessing data corresponding to a plurality of biological samples, the accessed data including a nucleotide sequence of a pathogen for each of the plurality of biological samples;
generating, based on the accessed data, an interactive dendrogram comprising an interactive portion that depicts a set of user-interface elements, wherein each user-interface element of the set of user-interface elements represents a biological sample of the plurality of biological samples, and wherein the user-interface elements in the set are arranged within the interactive portion based on a similarity of the nucleotide sequences of the biological samples that are respectively represented by the set of user-interface elements;
causing a graphical user interface to display the interactive dendrogram;
receiving, via the interactive portion of the graphical user interface, a selection of a user-interface element corresponding to a first biological sample;
identifying a first subset of biological samples associated with the first biological sample corresponding to the user-interface element, wherein each biological sample of the first subset is identified based on a determination that a number of variations between the nucleotide sequences of the biological samples of the first subset and the first biological sample is within a threshold; and
causing the graphical user interface to visually indicate user-interface elements that correspond to the biological samples in the subset.

8. The computer-implemented method of claim 7, further comprising:
receiving, via another interactive portion of the graphical user interface, an indication to update a first value corresponding to the threshold to a second value; and
updating the threshold from the first value to the second value.

9. The computer-implemented method of claim 8, wherein the other interactive portion of the graphical user interface includes a range-slider user-interface element.

10. The computer-implemented method of claim 7, wherein the threshold indicates a value corresponding to an extent of variations between nucleotide sequences of two biological samples.

11. The computer-implemented method of claim 10, further comprising:
updating the threshold from a first value to a second value, the second value being greater than the first value; and
as a result of updating the threshold from the first value to the second value, identifying a second subset of biological samples, wherein a number of the biological samples included the second subset is greater than a number of the biological samples included the first subset.

12. The computer-implemented method of claim 10, further comprising:
updating the threshold from a first value to a second value, the second value being less than the first value; and
as a result of updating the threshold from the first value to the second value, identifying a second subset of biological samples, wherein a number of the biological samples included the second subset is less than a number of the biological samples included the first subset.

13. The computer-implemented method of claim 7, wherein:
the first user-interface element is visually indicated with a first color; and
each of the user-interface elements that correspond to the biological samples in the subset is visually indicated with a second color.

14. A computer-implemented method for facilitating pathogen analysis of biological samples, the method comprising:
receiving, via a graphical user interface, a selection of two biological samples including different pathogens;
identifying, for each of the two biological samples, a nucleotide sequence;
generating a similarity matrix comprising a matrix element for each of the selected two biological samples, the matrix element indicating a number of variations between the identified nucleotide sequences of the two biological samples;
causing the graphical user interface to display the similarity matrix;
receiving, via the graphical user interface, another selection of a third biological sample;
identifying a third nucleotide sequence for the third biological sample;
transforming the similarity matrix by adding two other matrix elements, wherein each of the two other matrix elements indicate a number of variations between the third nucleotide sequence of the third biological sample and the nucleotide sequence of one of the two biological samples; and
causing the graphical user interface to display the transformed similarity matrix.

15. The computer-implemented method of claim 14, wherein a biological sample of the two biological samples is automatically selected based on a determination that variation between nucleotide sequences of biological sample and another biological sample of the two biological samples is within a predetermined single-nucleotide-polymorphism (SNP) threshold.

16. The computer-implemented method of claim 14, further comprising retrieving, from a database, a pre-computed value corresponding to the number of variations between the identified nucleotide sequences of the two biological samples.

17. The computer-implemented method of claim 16, wherein the database stores data corresponding to a plurality of biological samples, where a biological sample of the plurality of biological samples is associated with a set of pre-computed values, each pre-computed value of the set of pre-computed values corresponding to a number of variations between nucleotide sequences of the biological sample and another biological sample of the plurality of biological samples.

18. The computer-implemented method of claim 14, further comprising:
receiving, via a graphical user interface, a third selection of one of the three biological samples of the transformed similarity matrix; and
removing, from the transformed similarity matrix, matrix elements corresponding to the biological sample associated with the third selection.

19. A computer-implemented method of restricting access to information of biological samples, the method comprising:
receiving, at a server over a network, data from a group of users authorized to access a database, wherein the data correspond to a biological sample collected from a geographic region, and wherein the database comprises database records corresponding to a plurality of biological samples and nucleotide sequences corresponding to each of the plurality of biological samples;
processing the received data to identify a nucleotide sequence corresponding to the biological sample;
generating a new database record comprising an identifier representative of the biological sample and the nucleotide sequence of the biological sample;
storing the new database record into the database;
receiving, from a user, a request to access the new database record;
accessing a first identifier corresponding to the user, the first identifier indicating a first group of users affiliated with the user;
determining, based on the first identifier, that the first group and the authorized group of users do not match;
in response to determining that the first group and the authorized group do not match, redacting a first part of the new database record to generate a first redacted database record; and
providing, to the user, access of the database having the first redacted database record.

20. The computer-implemented method of claim 19, wherein the data corresponding to a biological sample further indicates a collection of user groups authorized to access the database, the method further comprising:
identifying, based on information of the first identifier, a second identifier corresponding to the user, the second identifier indicating a first collection of user groups corresponding to the first group;
determining, based on the second identifier, that the first collection of user groups and the authorized collection of user groups do not match;
in response to determining that the first collection of user groups and the authorized collection of user groups do not match, removing a second part of the new database record to generate a second redacted database record; and
providing, to the user, access of the database having the second redacted database record.

21. The computer-implemented method of claim 19, wherein the first redacted database record includes one or more anonymized parts of the new database record.

22. The computer-implemented method of claim 19, wherein redacting the part of the new database record includes replacing the first part of the new database record with information that prevents disclosure of the first part of the new database record.

23. The computer-implemented method of claim 19, wherein accessing the first identifier corresponding to the user includes retrieving the first identifier from registration information corresponding to the user.

24. The computer-implemented method of claim 19, wherein the database records of the database indicates a set of geographic regions, each of the set of geographic regions indicating a geographic region from which the biological sample of the plurality of biological samples was collected.

25-29. (canceled)
